SEQUENCE LISTING (6 sequences)

SEQ ID NO: 1 - protein, 1368 aa, synthetic construct [sequence omitted]

SEQ ID NO: 2 - DNA, 4251 bp, synthetic construct
  misc_feature 1..4251: note = SYNTHETIC
  source 1..4251: mol_type = other DNA
atgggcagcagcgccccaaagaagaagcggaaggtcggtatccacggagtcccagcagccatggacaaaaagtactctattggcctggatatcgggaccaacagcgtcgggtgggctgttatcaccgacgagtataaagtaccttcgaaaaagttcaaagtgctgggcaacaccgatcgccattcaatcaaaaagaacttgattggtgcgctgttgtttgactccggggaaaccgccgaggcgactcgccttaaacgtacagcacgtcgccggtacactcggcgtaagaatcgcatttgctatttgcaggaaatctttagcaacgagatggcaaaagtcgatgactcgtttttccaccgcctcgaggaaagctttctggtggaggaagacaaaaagcatgagcgtcacccgatcttcggcaacattgtcgatgaagtagcgtatcatgaaaaatacccaaccatttaccacttacgcaaaaagctggtggacagcactgacaaagctgatttgcgccttatctatttagccctggcacatatgattaagtttcgtggtcacttcctgatcgaaggagacttaaatcccgacaacagtgatgttgataaattgtttattcagcttgtccaaacttacaatcaactgttcgaggaaaacccgatcaatgcctccggtgtggatgcaaaagccattttaagtgcacgccttagcaagtcccgtcgcttagaaaaccttatcgcgcagctgcccggcgagaaaaagaatggtttgtttgggaaccttattgccttgagcttaggcctcaccccgaatttcaaaagtaatttcgatcttgcagaagacgccaaattacaactgtcgaaggatacttatgatgacgatctcgataatctgttagcgcagattggtgaccaatacgccgatctttttctggcggctaaaaatctgagcgacgccatcttgctttcggatattctccgcgttaacaccgaaatcacgaaagcgcctcttagtgccagcatgattaaacgttatgatgaacaccaccaggacctgaccttactcaaagcgttggttcgccagcaactgccagagaagtacaaagaaatcttctttgatcagtcaaagaatggttatgccggctatattgacgggggtgcaagccaagaggaattctacaaatttatcaagcctattctggagaaaatggatggcaccgaagagttattggtgaagcttaaccgtgaagacctcctgcggaaacagcgcacattcgataatggttcgatcccacaccaaatccatttgggggagttacacgctattttgcgtcgccaggaagacttttaccctttcctgaaggataaccgggagaaaattgagaagatccttacctttcgtattccgtattacgtaggccccttagcacggggtaatagccgtttcgcgtggatgacacggaagtcggaagagacgatcaccccgtggaacttcgaagaggtagtcgacaagggcgcatcagcgcagtcttttattgaacgtatgacgaatttcgataaaaacttgcccaatgagaaggtgcttccgaaacattccttgttatatgaatattttacagtttacaacgagctgaccaaggttaaatacgtgacggaaggaatgcgcaagcccgcttttcttagcggtgagcaaaaaaaggcgatcgtcgacctgttattcaaaacgaatcgtaaggtgactgtaaagcaactcaaagaagattacttcaaaaagattgagtgcttcgacagcgtcgaaatctctggggtagaggatcggtttaacgcaagtttaggtacctaccatgacctgcttaaaatcattaaggataaagacttcttagataatgaagagaacgaagatattctcgaggacatcgtcttgacgttaaccttatttgaggatcgtgaaatgattgaggaacgcctcaaaacttatgcccacctgttcgacgataaggtgatgaagcagctgaaacgtcggcgctacacaggatggggccgcttgagtcgcaaacttattaacggaatccgtgacaagcaatccggcaaaacgattctggatttcttgaagtcggacggatttgctaatcgcaacttcatgcagttgatccatgatgactccctgacttttaaagaggatattcaaaaggcgcaggttagtggtcaaggcgacagcttacacgaacacatcgcaaatttggctggttcgccggccattaaaaaggggatcctccagaccgtgaaagttgtagatgagcttgttaaggtcatgggtcgtcataagcccgaaaacatcgtgattgaaatggcgcgggagaatcaaacgacccagaaaggacaaaagaatagccgtgaacggatgaagcggatcgaggaaggcattaaagagctggggtctcaaatcttgaaggaacaccctgtggagaacactcagctccaaaatgaaaaactttacctgtactatttgcagaacggacgcgatatgtacgtggaccaagagttggatattaatcggctgagtgactacgacgttgatcatatcgtcccgcagagcttcctcaaagacgattctattgacaataaggtactgacgcgctctgataaaaaccgtggtaagtcggacaacgtgccctccgaagaggttgtgaaaaagatgaaaaattattggcgccagcttttaaacgcgaagctgatcacacaacgtaaattcgataatttgaccaaggctgaacggggtggcctgagcgagttagataaggcaggatttattaaacgccagttagtggagactcgtcaaatcaccaaacatgtcgcgcagattttggacagccggatgaacaccaagtacgatgaaaatgacaaactgatccgtgaggtgaaagtcattactctgaagtccaaattagttagtgatttccggaaggactttcaattctacaaagtccgtgaaattaataactatcatcacgcacatgacgcgtacctgaatgcagtggttgggaccgcccttatcaagaaatatcctaagctggagtcggagtttgtctatggcgactataaggtatacgatgttcgcaaaatgattgcgaaatctgagcaggagatcggtaaggcaaccgcaaaatatttcttttactcaaacattatgaatttctttaagacagaaatcactctggccaacggggagattcgcaaacgtccgttgatcgaaacaaacggcgagactggcgaaattgtttgggacaaagggcgtgatttcgcgacggtgcgcaaggtactgagcatgcctcaagtcaatattgttaagaaaaccgaagtgcagacgggcgggttttccaaggaaagcatcttacccaaacgtaattcagataaacttattgcacgcaaaaaggactgggatccgaaaaagtatggaggcttcgacagtccaaccgtagcctactctgttctcgttgtagcgaaagtagaaaagggtaaatccaagaaactgaaatctgtcaaggagttgcttggaatcaccattatggagcgtagctccttcgagaagaacccgattgactttctggaagccaaaggatataaagaggtcaagaaagatcttatcattaagctgcctaagtattcactcttcgagctggaaaatggtcgtaaacgcatgctcgcttctgccggcgagttgcagaagggcaatgaattagcacttccatcaaagtacgttaacttcctgtatttggccagccattacgagaaactgaaggggtctccagaggacaacgaacagaaacaattatttgtagagcagcacaagcattatcttgatgaaatcattgagcaaatttccgaattcagtaaacgcgtaatcctggccgatgcaaacctcgacaaggtgctgagcgcttacaataagcatcgcgacaaacctatccgtgagcaggctgaaaatatcattcacctgttcacattaacgaacctgggcgctccggccgcttttaaatatttcgacacgacaatcgaccgtaagcgctataccagtacgaaagaagtgttggatgcgacccttattcaccagtcaattacaggattatatgagacccgtatcgaccttagccaattaggtggggatgcggccccgaagaaaaaacgcaaagtggatccgaagaaaaaacgcaaagtggcggccgcactcgagcaccaccaccaccaccactga

SEQ ID NO: 3 - DNA, 4290 bp, synthetic construct
  misc_feature 1..4290: note = SYNTHETIC
  source 1..4290: mol_type = other DNA
atgggcaagcccatccctaaccccctgttggggctggacagcaccgctcccaaaaagaaaaggaaggtgggcattcacggcgtgcctgcggccgacaaaaagtacagcatcggccttgatatcggcaccaatagcgtgggctgggccgttatcacagacgaatacaaggtacccagcaagaagttcaaggtgctggggaatacagacaggcactctatcaagaaaaaccttatcggggctctgctgtttgactcaggcgagaccgccgaggccaccaggttgaagaggaccgcaaggcgaaggtacacccggaggaagaacaggatctgctatctgcaggagatcttcagcaacgagatggccaaggtggacgacagcttcttccacaggctggaggagagcttccttgtcgaggaggataagaagcacgaacgacaccccatcttcggcaacatagtcgacgaggtcgcttatcacgagaagtaccccaccatctaccacctgcgaaagaaattggtggatagcaccgataaagccgacttgcgacttatctacttggctctggcgcacatgattaagttcaggggccacttcctgatcgagggcgaccttaaccccgacaacagtgacgtagacaaattgttcatccagcttgtacagacctataaccagctgttcgaggaaaaccctattaacgccagcggggtggatgcgaaggccatacttagcgccaggctgagcaaaagcaggcgcttggagaacctgatagcccagctgcccggtgaaaagaagaacggcctcttcggtaatctgattgccctgagcctgggcctgacccccaacttcaagagcaacttcgacctggcagaagatgccaagctgcagttgagtaaggacacctatgacgacgacttggacaatctgctcgcccaaatcggcgaccagtacgctgacctgttcctcgccgccaagaacctttctgacgcaatcctgcttagcgatatccttagggtgaacacagagatcaccaaggcccccctgagcgccagcatgatcaagaggtacgacgagcaccatcaggacctgacccttctgaaggccctggtgaggcagcaactgcccgagaagtacaaggagatctttttcgaccagagcaagaacggctacgccggctacatcgacggcggagccagccaagaggagttctacaagttcatcaagcccatcctggagaagatggatggcaccgaggagctgctggtgaagctgaacagggaagatttgctccggaagcagaggacctttgacaacggtagcatcccccaccagatccacctgggcgagctgcacgcaatactgaggcgacaggaggatttctaccccttcctcaaggacaatagggagaaaatcgaaaagattctgaccttcaggatcccctactacgtgggccctcttgccaggggcaacagccgattcgcttggatgacaagaaagagcgaggagaccatcaccccctggaacttcgaggaagtggtggacaaaggagcaagcgcgcagtctttcatcgaacggatgaccaatttcgacaaaaacctgcctaacgagaaggtgctgcccaagcacagcctgctttacgagtacttcaccgtgtacaacgagctcaccaaggtgaaatatgtgaccgagggcatgcgaaaacccgctttcctgagcggcgagcagaagaaggccatcgtggacctgctgttcaagaccaacaggaaggtgaccgtgaagcagctgaaggaggactacttcaagaagatcgagtgctttgatagcgtggaaataagcggcgtggaggacaggttcaacgccagcctgggcacctaccacgacttgttgaagataatcaaagacaaggatttcctggataatgaggagaacgaggatatactcgaggacatcgtgctgactttgaccctgtttgaggaccgagagatgattgaagaaaggctcaaaacctacgcccacctgttcgacgacaaagtgatgaaacaactgaagagacgaagatacaccggctggggcagactgtccaggaagctcatcaacggcattagggacaagcagagcggcaagaccatcctggatttcctgaagtccgacggcttcgccaaccgaaacttcatgcagctgattcacgatgacagcttgaccttcaaggaggacatccagaaggcccaggttagcggccagggcgactccctgcacgaacatattgcaaacctggcaggctcccctgcgatcaagaagggcatactgcagaccgttaaggttgtggacgaattggtcaaggtcatgggcaggcacaagcccgaaaacatagttatagagatggccagagagaaccagaccacccaaaagggccagaagaacagccgggagcgcatgaaaaggatcgaggagggtatcaaggaactcggaagccagatcctcaaagagcaccccgtggagaatacccagctccagaacgagaagctgtacctgtactacctgcagaacggcagggacatgtacgttgaccaggagttggacatcaacaggctttcagactatgacgtggatcacatagtgccccagagctttcttaaagacgatagcatcgacaacaaggtcctgacccgctccgacaaaaacaggggcaaaagcgacaacgtgccaagcgaagaggtggttaaaaagatgaagaactactggaggcaactgctcaacgcgaaattgatcacccagagaaagttcgataacctgaccaaggccgagaggggcggactctccgaacttgacaaagcgggcttcataaagaggcagctggtcgagacccgacagatcacgaagcacgtggcccaaatcctcgacagcagaatgaataccaagtacgatgagaatgacaaactcatcagggaagtgaaagtgattaccctgaagagcaagttggtgtccgactttcgcaaagatttccagttctacaaggtgagggagatcaacaactaccaccatgcccacgacgcatacctgaacgccgtggtcggcaccgccctgattaagaagtatccaaagctggagtccgaatttgtctacggcgactacaaagtttacgatgtgaggaagatgatcgctaagagcgaacaggagatcggcaaggccaccgctaagtatttcttctacagcaacatcatgaactttttcaagaccgagatcacacttgccaacggcgaaatcaggaagaggccgcttatcgagaccaacggtgagaccggcgagatcgtgtgggacaagggcagggacttcgccaccgtgaggaaagtcctgagcatgccccaggtgaatattgtgaaaaaaactgaggtgcagacaggcggctttagcaaggaatccatcctgcccaagaggaacagcgacaagctgatcgcccggaagaaggactgggaccctaagaagtatggaggcttcgacagccccaccgtagcctacagcgtgctggtggtcgcgaaggtagagaaggggaagagcaagaaactgaagagcgtgaaggagctgctcggcataaccatcatggagaggtccagctttgagaagaaccccattgactttttggaagccaagggctacaaagaggtcaaaaaggacctgatcatcaaactccccaagtactccctgtttgaattggagaacggcagaaagaggatgctggcgagcgctggggaactgcaaaagggcaacgaactggcgctgcccagcaagtacgtgaattttctgtacctggcgtcccactacgaaaagctgaaaggcagccccgaggacaacgagcagaagcagctgttcgtggagcagcacaagcattacctggacgagataatcgagcaaatcagcgagttcagcaagagggtgattctggccgacgcgaacctggataaggtcctcagcgcctacaacaagcaccgagacaaacccatcagggagcaggccgagaatatcatacacctgttcaccctgacaaatctgggcgcacctgcggcattcaaatacttcgataccaccatcgacaggaaaaggtacactagcactaaggaggtgctggatgccaccttgatccaccagtccattaccggcctgtatgagaccaggatcgacctgagccagcttggaggcgactctagggcggacccaaaaaagaaaaggaaggtggaattccaccacactggactagtggatccgagctcggtaccaagcttaagtttaaaccgctga

SEQ ID NO: 4 - protein, 1368 aa, synthetic construct
  REGION 1..1368: note = SYNTHETIC
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 5 - DNA, 4251 bp, synthetic construct
  misc_feature 1..4251: note = SYNTHETIC
  source 1..4251: mol_type = other DNA
atgggcagcagcgccccaaagaagaagcggaaggtcggtatccacggagtcccagcagccatggacaaaaagtactctattggcctggatatcgggaccaacagcgtcgggtgggctgttatcaccgacgagtataaagtaccttcgaaaaagttcaaagtgctgggcaacaccgatcgccattcaatcaaaaagaacttgattggtgcgctgttgtttgactccggggaaaccgccgaggcgactcgccttaaacgtacagcacgtcgccggtacactcggcgtaagaatcgcatttgctatttgcaggaaatctttagcaacgagatggcaaaagtcgatgactcgtttttccaccgcctcgaggaaagctttctggtggaggaagacaaaaagcatgagcgtcacccgatcttcggcaacattgtcgatgaagtagcgtatcatgaaaaatacccaaccatttaccacttacgcaaaaagctggtggacagcactgacaaagctgatttgcgccttatctatttagccctggcacatatgattaagtttcgtggtcacttcctgatcgaaggagacttaaatcccgacaacagtgatgttgataaattgtttattcagcttgtccaaacttacaatcaactgttcgaggaaaacccgatcaatgcctccggtgtggatgcaaaagccattttaagtgcacgccttagcaagtcccgtcgcttagaaaaccttatcgcgcagctgcccggcgagaaaaagaatggtttgtttgggaaccttattgccttgagcttaggcctcaccccgaatttcaaaagtaatttcgatcttgcagaagacgccaaattacaactgtcgaaggatacttatgatgacgatctcgataatctgttagcgcagattggtgaccaatacgccgatctttttctggcggctaaaaatctgagcgacgccatcttgctttcggatattctccgcgttaacaccgaaatcacgaaagcgcctcttagtgccagcatgattaaacgttatgatgaacaccaccaggacctgaccttactcaaagcgttggttcgccagcaactgccagagaagtacaaagaaatcttctttgatcagtcaaagaatggttatgccggctatattgacgggggtgcaagccaagaggaattctacaaatttatcaagcctattctggagaaaatggatggcaccgaagagttattggtgaagcttaaccgtgaagacctcctgcggaaacagcgcacattcgataatggttcgatcccacaccaaatccatttgggggagttacacgctattttgcgtcgccaggaagacttttaccctttcctgaaggataaccgggagaaaattgagaagatccttacctttcgtattccgtattacgtaggccccttagcacggggtaatagccgtttcgcgtggatgacacggaagtcggaagagacgatcaccccgtggaacttcgaagaggtagtcgacaagggcgcatcagcgcagtcttttattgaacgtatgacgaatttcgataaaaacttgcccaatgagaaggtgcttccgaaacattccttgttatatgaatattttacagtttacaacgagctgaccaaggttaaatacgtgacggaaggaatgcgcaagcccgcttttcttagcggtgagcaaaaaaaggcgatcgtcgacctgttattcaaaacgaatcgtaaggtgactgtaaagcaactcaaagaagattacttcaaaaagattgagtgcttcgacagcgtcgaaatctctggggtagaggatcggtttaacgcaagtttaggtacctaccatgacctgcttaaaatcattaaggataaagacttcttagataatgaagagaacgaagatattctcgaggacatcgtcttgacgttaaccttatttgaggatcgtgaaatgattgaggaacgcctcaaaacttatgcccacctgttcgacgataaggtgatgaagcagctgaaacgtcggcgctacacaggatggggccgcttgagtcgcaaacttattaacggaatccgtgacaagcaatccggcaaaacgattctggatttcttgaagtcggacggatttgctaatgcgaacttcatgcagttgatccatgatgactccctgacttttaaagaggatattcaaaaggcgcaggttagtggtcaaggcgacagcttacacgaacacatcgcaaatttggctggttcgccggccattaaaaaggggatcctccagaccgtgaaagttgtagatgagcttgttaaggtcatgggtcgtcataagcccgaaaacatcgtgattgaaatggcgcgggagaatcaaacgacccagaaaggacaaaagaatagccgtgaacggatgaagcggatcgaggaaggcattaaagagctggggtctcaaatcttgaaggaacaccctgtggagaacactcagctccaaaatgaaaaactttacctgtactatttgcagaacggacgcgatatgtacgtggaccaagagttggatattaatcggctgagtgactacgacgttgatcatatcgtcccgcagagcttcctcaaagacgattctattgacaataaggtactgacgcgctctgataaaaaccgtggtaagtcggacaacgtgccctccgaagaggttgtgaaaaagatgaaaaattattggcgccagcttttaaacgcgaagctgatcacacaacgtaaattcgataatttgaccaaggctgaacggggtggcctgagcgagttagataaggcaggatttattaaacgccagttagtggagactcgtcaaatcaccaaacatgtcgcgcagattttggacagccggatgaacaccaagtacgatgaaaatgacaaactgatccgtgaggtgaaagtcattactctgaagtccaaattagttagtgatttccggaaggactttcaattctacaaagtccgtgaaattaataactatcatcacgcacatgacgcgtacctgaatgcagtggttgggaccgcccttatcaagaaatatcctaagctggagtcggagtttgtctatggcgactataaggtatacgatgttcgcaaaatgattgcgaaatctgagcaggagatcggtaaggcaaccgcaaaatatttcttttactcaaacattatgaatttctttaagacagaaatcactctggccaacggggagattcgcaaacgtccgttgatcgaaacaaacggcgagactggcgaaattgtttgggacaaagggcgtgatttcgcgacggtgcgcaaggtactgagcatgcctcaagtcaatattgttaagaaaaccgaagtgcagacgggcgggttttccaaggaaagcatcttacccaaacgtaattcagataaacttattgcacgcaaaaaggactgggatccgaaaaagtatggaggcttcgacagtccaaccgtagcctactctgttctcgttgtagcgaaagtagaaaagggtaaatccaagaaactgaaatctgtcaaggagttgcttggaatcaccattatggagcgtagctccttcgagaagaacccgattgactttctggaagccaaaggatataaagaggtcaagaaagatcttatcattaagctgcctaagtattcactcttcgagctggaaaatggtcgtaaacgcatgctcgcttctgccggcgagttgcagaagggcaatgaattagcacttccatcaaagtacgttaacttcctgtatttggccagccattacgagaaactgaaggggtctccagaggacaacgaacagaaacaattatttgtagagcagcacaagcattatcttgatgaaatcattgagcaaatttccgaattcagtaaacgcgtaatcctggccgatgcaaacctcgacaaggtgctgagcgcttacaataagcatcgcgacaaacctatccgtgagcaggctgaaaatatcattcacctgttcacattaacgaacctgggcgctccggccgcttttaaatatttcgacacgacaatcgaccgtaagcgctataccagtacgaaagaagtgttggatgcgacccttattcaccagtcaattacaggattatatgagacccgtatcgaccttagccaattaggtggggatgcggccccgaagaaaaaacgcaaagtggatccgaagaaaaaacgcaaagtggcggccgcactcgagcaccaccaccaccaccactga

SEQ ID NO: 6 - DNA, 4290 bp, synthetic construct
  misc_feature 1..4290: note = SYNTHETIC
  source 1..4290: mol_type = other DNA
atgggcaagcccatccctaaccccctgttggggctggacagcaccgctcccaaaaagaaaaggaaggtgggcattcacggcgtgcctgcggccgacaaaaagtacagcatcggccttgatatcggcaccaatagcgtgggctgggccgttatcacagacgaatacaaggtacccagcaagaagttcaaggtgctggggaatacagacaggcactctatcaagaaaaaccttatcggggctctgctgtttgactcaggcgagaccgccgaggccaccaggttgaagaggaccgcaaggcgaaggtacacccggaggaagaacaggatctgctatctgcaggagatcttcagcaacgagatggccaaggtggacgacagcttcttccacaggctggaggagagcttccttgtcgaggaggataagaagcacgaacgacaccccatcttcggcaacatagtcgacgaggtcgcttatcacgagaagtaccccaccatctaccacctgcgaaagaaattggtggatagcaccgataaagccgacttgcgacttatctacttggctctggcgcacatgattaagttcaggggccacttcctgatcgagggcgaccttaaccccgacaacagtgacgtagacaaattgttcatccagcttgtacagacctataaccagctgttcgaggaaaaccctattaacgccagcggggtggatgcgaaggccatacttagcgccaggctgagcaaaagcaggcgcttggagaacctgatagcccagctgcccggtgaaaagaagaacggcctcttcggtaatctgattgccctgagcctgggcctgacccccaacttcaagagcaacttcgacctggcagaagatgccaagctgcagttgagtaaggacacctatgacgacgacttggacaatctgctcgcccaaatcggcgaccagtacgctgacctgttcctcgccgccaagaacctttctgacgcaatcctgcttagcgatatccttagggtgaacacagagatcaccaaggcccccctgagcgccagcatgatcaagaggtacgacgagcaccatcaggacctgacccttctgaaggccctggtgaggcagcaactgcccgagaagtacaaggagatctttttcgaccagagcaagaacggctacgccggctacatcgacggcggagccagccaagaggagttctacaagttcatcaagcccatcctggagaagatggatggcaccgaggagctgctggtgaagctgaacagggaagatttgctccggaagcagaggacctttgacaacggtagcatcccccaccagatccacctgggcgagctgcacgcaatactgaggcgacaggaggatttctaccccttcctcaaggacaatagggagaaaatcgaaaagattctgaccttcaggatcccctactacgtgggccctcttgccaggggcaacagccgattcgcttggatgacaagaaagagcgaggagaccatcaccccctggaacttcgaggaagtggtggacaaaggagcaagcgcgcagtctttcatcgaacggatgaccaatttcgacaaaaacctgcctaacgagaaggtgctgcccaagcacagcctgctttacgagtacttcaccgtgtacaacgagctcaccaaggtgaaatatgtgaccgagggcatgcgaaaacccgctttcctgagcggcgagcagaagaaggccatcgtggacctgctgttcaagaccaacaggaaggtgaccgtgaagcagctgaaggaggactacttcaagaagatcgagtgctttgatagcgtggaaataagcggcgtggaggacaggttcaacgccagcctgggcacctaccacgacttgttgaagataatcaaagacaaggatttcctggataatgaggagaacgaggatatactcgaggacatcgtgctgactttgaccctgtttgaggaccgagagatgattgaagaaaggctcaaaacctacgcccacctgttcgacgacaaagtgatgaaacaactgaagagacgaagatacaccggctggggcagactgtccaggaagctcatcaacggcattagggacaagcagagcggcaagaccatcctggatttcctgaagtccgacggcttcgccaacgccaacttcatgcagctgattcacgatgacagcttgaccttcaaggaggacatccagaaggcccaggttagcggccagggcgactccctgcacgaacatattgcaaacctggcaggctcccctgcgatcaagaagggcatactgcagaccgttaaggttgtggacgaattggtcaaggtcatgggcaggcacaagcccgaaaacatagttatagagatggccagagagaaccagaccacccaaaagggccagaagaacagccgggagcgcatgaaaaggatcgaggagggtatcaaggaactcggaagccagatcctcaaagagcaccccgtggagaatacccagctccagaacgagaagctgtacctgtactacctgcagaacggcagggacatgtacgttgaccaggagttggacatcaacaggctttcagactatgacgtggatcacatagtgccccagagctttcttaaagacgatagcatcgacaacaaggtcctgacccgctccgacaaaaacaggggcaaaagcgacaacgtgccaagcgaagaggtggttaaaaagatgaagaactactggaggcaactgctcaacgcgaaattgatcacccagagaaagttcgataacctgaccaaggccgagaggggcggactctccgaacttgacaaagcgggcttcataaagaggcagctggtcgagacccgacagatcacgaagcacgtggcccaaatcctcgacagcagaatgaataccaagtacgatgagaatgacaaactcatcagggaagtgaaagtgattaccctgaagagcaagttggtgtccgactttcgcaaagatttccagttctacaaggtgagggagatcaacaactaccaccatgcccacgacgcatacctgaacgccgtggtcggcaccgccctgattaagaagtatccaaagctggagtccgaatttgtctacggcgactacaaagtttacgatgtgaggaagatgatcgctaagagcgaacaggagatcggcaaggccaccgctaagtatttcttctacagcaacatcatgaactttttcaagaccgagatcacacttgccaacggcgaaatcaggaagaggccgcttatcgagaccaacggtgagaccggcgagatcgtgtgggacaagggcagggacttcgccaccgtgaggaaagtcctgagcatgccccaggtgaatattgtgaaaaaaactgaggtgcagacaggcggctttagcaaggaatccatcctgcccaagaggaacagcgacaagctgatcgcccggaagaaggactgggaccctaagaagtatggaggcttcgacagccccaccgtagcctacagcgtgctggtggtcgcgaaggtagagaaggggaagagcaagaaactgaagagcgtgaaggagctgctcggcataaccatcatggagaggtccagctttgagaagaaccccattgactttttggaagccaagggctacaaagaggtcaaaaaggacctgatcatcaaactccccaagtactccctgtttgaattggagaacggcagaaagaggatgctggcgagcgctggggaactgcaaaagggcaacgaactggcgctgcccagcaagtacgtgaattttctgtacctggcgtcccactacgaaaagctgaaaggcagccccgaggacaacgagcagaagcagctgttcgtggagcagcacaagcattacctggacgagataatcgagcaaatcagcgagttcagcaagagggtgattctggccgacgcgaacctggataaggtcctcagcgcctacaacaagcaccgagacaaacccatcagggagcaggccgagaatatcatacacctgttcaccctgacaaatctgggcgcacctgcggcattcaaatacttcgataccaccatcgacaggaaaaggtacactagcactaaggaggtgctggatgccaccttgatccaccagtccattaccggcctgtatgagaccaggatcgacctgagccagcttggaggcgactctagggcggacccaaaaaagaaaaggaaggtggaattccaccacactggactagtggatccgagctcggtaccaagcttaagtttaaaccgctga

(19) United States
(12) Patent Application Publication
(10) Pub. No.: US 2025/0320508 A1
(43) Pub. Date: Oct. 16, 2025

(54) Novel Mutations in Streptococcus Pyogenes CAS9 Discovered by Broad Scanning Mutagenesis Demonstrate Enhancement of DNA Cleavage Activity

(71) Applicant: Integrated DNA Technologies, Inc., Coralville, IA (US)

(72) Inventors: Nathaniel Hunter ROBERTS, Iowa City, IA (US); Liyang ZHANG, North Liberty, IA (US); Christopher Anthony Vakulskas, North Liberty, IA (US)

(21) Appl. No.: 19/182,953

(22) Filed: Apr. 18, 2025

Related U.S. Application Data
(63) Continuation of application No. 17/857,262, filed on Jul. 5, 2022, now Pat. No. 12,297,435.
(60) Provisional application No. 63/217,881, filed on Jul. 2, 2021.

(51) Int. Cl. C12N 15/52; C12N 9/22; C12N 15/11

(52) U.S. Cl. CPC C12N 15/52; C12N 9/22; C12N 15/111; C12N 2310/20

(57) ABSTRACT

This invention pertains to mutant Cas9 nucleic acids and proteins for use in CRISPR/Cas endonuclease systems, and their methods of use. In particular, the invention pertains to an isolated mutant Cas9 protein, wherein the isolated mutant Cas9 protein is active in a CRISPR/Cas endonuclease system, wherein the CRISPR/Cas endonuclease system displays increased on-target editing activity relative to a wild-type CRISPR/Cas endonuclease system. The invention also includes isolated nucleic acids encoding mutant Cas9 proteins, ribonucleoprotein complexes and CRSPR/Cas endonuclease systems having mutant Cas9 proteins that display increased on-target editing activity relative to a wild-type CRISPR/Cas endonuclease system.

### CROSS-REFERENCE TO RELATED APPLICATIONS

This application is Continuation of U.S. application Ser. No. 17/857,262, filed Jul. 5, 2022, which claims benefit of priority under 35 U.S.C. 119 to U.S. Provisional Patent Application Ser. No. 63/217,881, filed Jul. 2, 2021 and entitled “NOVEL MUTATIONS IN STREPTOCOCCUS PYOGENES CAS9 DISCOVERED BY BROAD SCANNING MUTAGENESIS DEMONSTRATE ENHANCEMENT OF DNA CLEAVAGE ACTIVITY,” the contents of each application are herein incorporated by reference in its entirety.

### SEQUENCE LISTING

The instant application contains Sequence Listings which have been submitted in XML format via Patent Center and are hereby incorporated by reference in their entireties. Said XML copies, created on Apr. 18, 2025, are named 6391-0017US02_A.xml, which is 28 k kbytes in size, and 6391-0017US02_B, which is 611 kbytes in size.

### FIELD OF THE INVENTION

This invention pertains to Cas9 mutant genes, polypeptides encoded by the same and their use in compositions of CRISPR-Cas systems for improving on-site targeted cleavage activity.

### BACKGROUND OF THE INVENTION

SpCas9 is an RNA-guided endonuclease utilizing the Clustered Regularly Interspaced Short Palindromic Repeat (CRISPR) adaptive immune system from Streptococcus pyogenes. Cas9 utilizes a RNA guide complementary to a 20 nucleotide DNA target sequence, commonly referred to as a protospacer. Critical to the recognition of correct DNA target for SpCas9 includes both crRNA and the canonical “NGG” protospacer adjacent motif (PAM), which is a 3-bp sequence immediately downstream of the protospacer. The Cas9-gRNA ribonucleoprotein (RNP) complex mediates double-stranded DNA breaks (DSBs), which are then repaired by either the non-homologous end joining (NHEJ, typically introduces mutations or indels at the cut site), or the homology directed repair (HDR) system for precise editing if a suitable template nucleic acid is present.

Currently, many point mutations have been identified to improve the specificity of SpCas9 to mitigate off-target editing, although at the significant cost of on-target potency^{[1]}. However, for applications where off-target editing is less of a concern (such as high-throughput genetic screen), a high-activity SpCas9 with uniform editing efficiency across all targetable sites is desirable. In addition, while many SpCas9 variants with alternative PAM preference have been developed, their utilities are limited to the reduced on-target potency^{[3]}. Increasing on-target activity of SpCas9 through mutagenesis may rescue the poor performance of these PAM variants in human cells when delivered as RNP. Further, it has been suggested that the on-target activity of SpCas9 is a rate-limiting step for advanced gene editing platforms established upon SpCas9, such base editing and prime editing.

Therefore, a need remains for methods to improve specificity of Cas9 genome editing. In particular, there exists a need to identify novel point mutations to enhance or increase on-target activity of SpCas9 to improve the performance of a variety of gene editing platforms that are widely adopted by the community^{[4]}. This disclosure provides a solution to a long-felt need of generating novel point mutations to enhance on-target activity of SpCas9 that may improve the performance of a variety of gene editing platforms that are widely adopted by the community^{[4]}.

### BRIEF SUMMARY OF THE INVENTION

This invention pertains to Cas9 mutant genes and polypeptides for use in CRISPR systems, and their methods of use.

In a first aspect, an isolated mutant Cas9 protein is provided. The isolated mutant Cas9 protein is active in a Clustered Regularly Interspaced Short Palindromic Repeats (CRISPR)/CRISPR-associated protein endonuclease system (“CRISPR/Cas endonuclease system”). The CRISPR/Cas endonuclease system displays increased on-target editing activity relative to a wild-type CRISPR/Cas endonuclease system.

In a second aspect, an isolated ribonucleoprotein (RNP) complex is provided. The RNP complex includes a mutant Cas9 protein and a gRNA complex. The isolated ribonucleoprotein complex is active as a CRISPR/Cas endonuclease system, wherein the resultant CRISPR/Cas endonuclease system displays increased on-target editing activity relative to a wild-type CRISPR/Cas endonuclease system.

In a third aspect, an isolated nucleic acid encoding a mutant Cas9 protein is provided. The mutant Cas9 protein is active in a CRISPR/Cas endonuclease system, wherein the CRISPR/Cas endonuclease system displays increased on-target editing activity relative to a wild-type CRISPR/Cas endonuclease system

In a fourth aspect, a CRISPR/Cas endonuclease system is provided. The CRISPR/Cas endonuclease system includes a mutant Cas9 protein and a gRNA. The CRISPR/Cas endonuclease system displays increased on-target editing activity relative to a wild-type CRISPR/Cas endonuclease system.

In a fifth aspect, a method of performing gene editing having increased on-target editing activity is provided. The method includes the step of contacting a candidate editing DNA target site locus with an active CRISPR/Cas endonuclease system having a mutant Cas9 protein complexed with an appropriate gRNA (e.g., crRNA:tracrRNA complex or sgRNA). Said interaction can occur in any context, for example, in a live animal, in live cells, or in isolated DNA in vitro.

### BRIEF DESCRIPTION OF THE DRAWINGS

FIG. 1 depicts an exemplary Pearson Correlation of natural log scale enriched mutations replicate 1 and replicate 2. This figure shows good agreement between replicates and indicates a fraction of SpCas9 mutants with increased cleavage activity.

FIG. 2 depicts examples of individual novel SpCas9 mutations that enhance DNA cleavage utilizing mismatched guide RNA. The cleavage assay shows a validation of the initial screening process in which mutants show higher survival rates under selection than Wild-Type SpCas9. Survival of E. coli was dependent on the ability of Cas9 to cleave the intended target site on the toxin-encoding plasmid. Panels A-M depict Wild-Type SpCas9 plasmid with ‘mismatch 15’ guide RNA (A); Wild-Type SpCas9 with perfectly-matched guide RNA (B); Wild-Type SpCas9 plasmid with no guide RNA (C): Panels D-M: SpCas9 polypeptides having mutant single amino acid mutations identified from mutagenesis screen with ‘mismatch 15’ guide RNA: SpCas9 D54W (D); SpCas9 D54K (E); SpCas9 N46L (F); SpCas9 S6T (G); SpCas9 T67D (H); SpCas9 S55N (I); SpCas9 D54A (J); SpCas9 R71W (K); SpCas9 A68D (L); and SpCas9 V16I (M).

FIG. 3A depicts exemplary SpCas9 mutants with enhanced on-target editing efficiency in human cells. Putative mutations with beneficial effect in E. coli activity assay was introduced in the background of Hifi-Cas9 (R691A). Mutant proteins were purified, and evaluated in HEK293 cells by editing three target sites. On-target editing efficiency of novel Cas9 mutants. The dot lines highlight the editing efficiency of reference protein (R691A) at each target.

FIG. 3B depicts normalized editing efficiency of each mutant against reference protein (R691A). Of note, D54A/K significantly elevated the on-target activity of R691A, even surpassing WT-SpCas9.

FIG. 3C depicts off-target editing of novel Cas9 mutants at EMX1 off-target site. Only WT-SpCas9 displayed any measurable off-target effect. All novel Cas9 mutants remained highly specific.

### DETAILED DESCRIPTION OF THE INVENTION

The methods and compositions of the invention described herein provide mutant SpyCas9 nucleic acids and polypeptides for use in a CRISPR-Cas system. The present invention describes novel Cas9 mutants that increased on-target editing activity relative to the wild-type protein. These and other advantages of the invention, as well as additional inventive features, will be apparent from the description of the invention provided herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “wild-type Cas9 protein” (“WT-Cas9” or “WT-Cas9 protein”) encompasses a protein having the identical amino acid sequence of the naturally-occurring Streptococcus pyogenes Cas9 (e.g., SEQ ID NO:1) and that has biochemical and biological activity when combined with a suitable guide RNA (for example sgRNA or dual crRNA:tracrRNA compositions) to form an active CRISPR-Cas endonuclease system.

The term “wild-type CRISPR/Cas endonuclease system” refers to a CRISPR/Cas endonuclease system that includes wild-type Cas9 protein and a suitable gRNA.

The phrase “active CRISPR/Cas endonuclease system displays increased on-target editing activity relative to a wild-type CRISPR/Cas endonuclease system” refers to the activity of a CRISPR/Cas endonuclease system that includes a mutant Cas9 protein that displays an enhanced or increased on-target editing activities of a wild-type CRISPR/Cas endonuclease system that includes wild-type Cas9 protein of SEQ ID NO: 1 when both CRISPR/Cas endonuclease systems include the identical gRNA for a given target sequence. Preferred on-target activities of the CRISPR/Cas endonuclease systems depend upon the gRNA and the target sequence of interest; such preferred increased on-target activities of CRISPR/Cas endonuclease systems having mutant Cas9 proteins are illustrated in the herein.

The term “polypeptide” refers to any linear or branched peptide comprising more than one amino acid. Polypeptide includes protein or fragment thereof or fusion thereof, provided such protein, fragment or fusion retains a useful biochemical or biological activity.

Fusion proteins typically include extra amino acid information that is not native to the protein to which the extra amino acid information is covalently attached. Such extra amino acid information may include tags that enable purification or identification of the fusion protein. Such extra amino acid information may include peptides that enable the fusion proteins to be transported into cells and/or transported to specific locations within cells.

The term “isolated nucleic acid” include DNA, RNA, cDNA, and vectors encoding the same, where the DNA, RNA, cDNA and vectors are free of other biological materials from which they may be derived or associated, such as cellular components. Typically, an isolated nucleic acid will be purified from other biological materials from which they may be derived or associated, such as cellular components.

The term “isolated wild-type Cas9 nucleic acid” is an isolated nucleic acid that encodes a wild-type Cas9 protein. Examples of an isolated wild-type Cas9 nucleic acid include codon-optimized versions designed for efficient expression in E. coli or human cells, such as those nucleic acids encoded by SEQ ID NO: 2 and 3.

The term “isolated mutant Cas9 nucleic acid” is an isolated nucleic acid that encodes a mutant Cas9 protein, such as a Cas9 protein having an R691A mutation (SEQ ID NO: 4). Examples of an isolated mutant Cas9 nucleic acid include codon-optimized versions designed for efficient expression in E. coli or human cells, such as those nucleic acids encoded by SEQ ID NO: 5 and 6, respectively.

The term “length-modified,” as that term modifies RNA, refers to a shortened or truncated form of a reference RNA lacking nucleotide sequences or an elongated form of a reference RNA including additional nucleotide sequences.

The term “chemically-modified,” as that term modifies RNA, refers to a form of a reference RNA containing a chemically-modified nucleotide or a non-nucleotide chemical group covalently linked to the RNA. Chemically-modified RNA, as described herein, generally refers to synthetic RNA prepared using oligonucleotide synthesis procedures wherein modified nucleotides are incorporated during synthesis of an RNA oligonucleotide. However, chemically-modified RNA also includes synthetic RNA oligonucleotides modified with suitable modifying agents post-synthesis.

The term ALT-R®, as that term modifies an RNA (for example, such as a crRNA, a tracrRNA, a guide RNA, or a sgRNA), refers to an isolated, chemically synthesized, synthetic RNA.

The term ALT-R®, as that term modifies a protein (for example, such as a Cas9 protein), refers to an isolated, recombinant protein.

A competent CRISPR-Cas endonuclease system includes a ribonucleoprotein (RNP) complex formed with isolated Cas9 protein and isolated guide RNA selected from one of a dual crRNA:tracrRNA combination or a chimeric single-molecule sgRNA. In some embodiments, isolated length-modified and/or chemically-modified forms of crRNA and tracrRNA are combined with purified Cas9 protein, an isolated mRNA encoding Cas9 protein or a gene encoding Cas9 protein in an expression vector. In certain assays, isolated length-modified and/or chemically-modified forms of crRNA and tracrRNA can be introduced into cell lines that stably express Cas9 protein from an endogenous expression cassette encoding the Cas9 gene. In other assays, a mixture of length-modified and/or chemically-modified forms of crRNA and tracrRNA in combination with either mutant Cas9 mRNA or mutant Cas9 protein can be introduced into cells.

### Mutant Cas9 Proteins Having Increased On-Target Gene Editing Activity

In a first aspect, an isolated mutant Cas9 protein is provided. The isolated mutant Cas9 protein is active in a Clustered Regularly Interspaced Short Palindromic Repeats (CRISPR)/CRISPR-associated protein endonuclease system (“CRISPR/Cas endonuclease system”). The resultant CRISPR/Cas endonuclease system displays increased on-target editing activity relative to a wild-type CRISPR/Cas endonuclease system.

A bacterial-based directed evolution of SpCas9 was performed to identify mutations with enhanced cleavage activity. A deep-scanning mutagenesis library containing all possible point mutations on the amino acid level over positions 1-75 of SpCas9 was initially created, with most clones containing only one mutation ^{[2]}. This type of library allows one to directly evaluate the phenotype of each point mutation, by measuring their relative survival rates over the wild-type (WT) protein in the bacterial screen. Since SpCas9 activity is generally very high in E. coli, a crRNA and target site with mismatches was selected to down-tune the cleavage activity, thereby creating a suitable selection pressure to identify mutant with enhanced activity (Table 1).

TABLE 1
RNA guides used for Cas9 screen.
Mismatch guide used for selection
in comparison to WT-guide RNA.

| | |
|---|---|
| Perfect Match | GGTGAGTGAGTGTGTGCGTG |
| MM7 | GGCCAGTGAGTGTGTGCGTG |
| MM15 | GGTGCCCGAGTGTGTGCGTG |

To identify high-activity mutant, the screening strain harboring the toxin plasmid was transformed with SpCas9 library and mismatched crRNA targeting a single site on the toxin plasmid. After recovery and induction, cells were plated on selective media with induction and incubated at 37° C. overnight. SpCas9 expression plasmids carried by the surviving E. coli cells were extracted and purified. Both input and selected plasmid libraries were sequenced with NGS to determine the frequencies of mutations at each position of SpCas9 in both libraries. The relative survival rate of each point mutation was calculated as the ratio of normalized frequency between selected and input library. Since the degree of cell survival under selection is indicative of the cleavage activity of SpCas9, any variants that are enriched during the selection over WT would be those with enhanced activity.

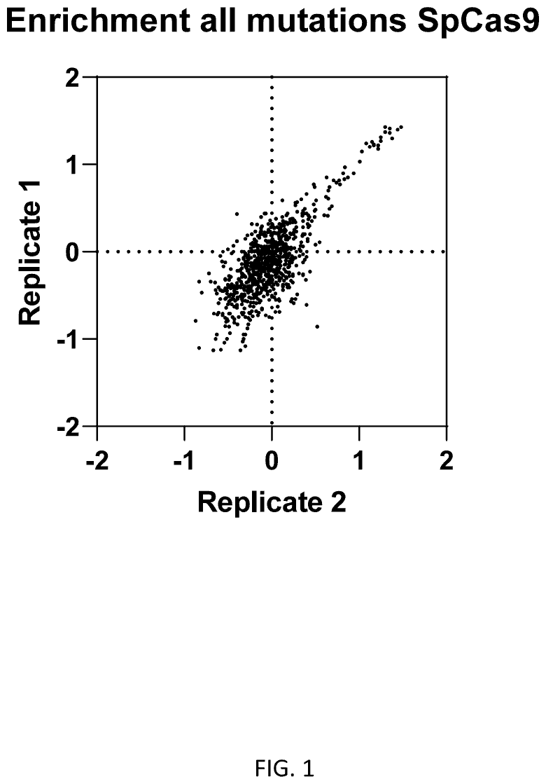

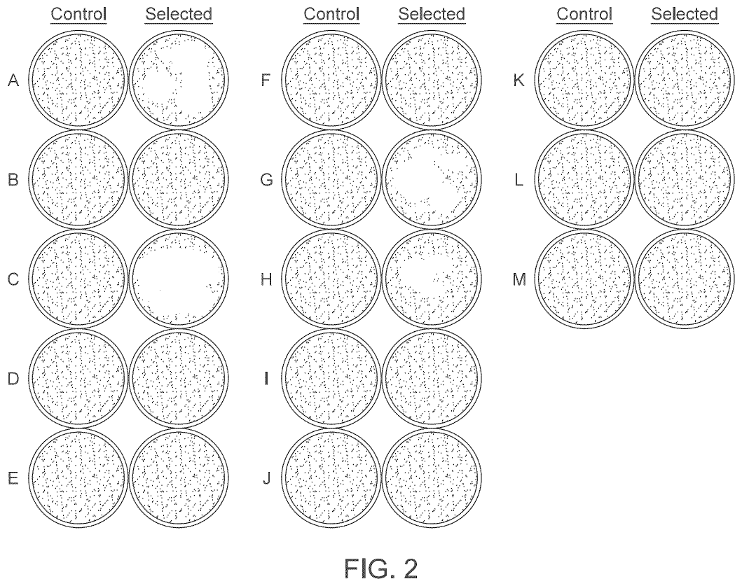

Table 2 and FIG. 1 summarize the phenotype of 266 point mutations of SpCas9 in the bacterial screen measuring the cleavage activity with a mismatched gRNA. Three biological replicates were performed with high consistency, which enabled us to confidently isolate a large collection of novel SpCas9 variants with enhanced activity. We therefore selectively validated several candidates in the context of E. coli cleavage assay. Individual mutant plasmid was cloned and delivered to screening E. coli strain in the presence of mismatched gRNA. Multiple novel SpCas9 mutants displayed significant improvement of DNA cleavage in E. coli over wild-type protein, which is indicated by a substantial increase of survival rate upon selection (FIG. 2). These results suggest the result of our high-throughput screen is highly accurate, which provided a large pool of candidates for further validation in the context of human cells.

TABLE 2
Point mutations enriched over WT SpCas9 (SEQ ID NO: 1). Shown
in natural log, phenotype enrichment scores demonstrate
point mutations of improving Cas9 activity.

| Num- | | | Wild-Type | Mutation | Aver- | Standard |
|---|---|---|---|---|---|---|
| ber | Mutation | Position | Residue | Residue | age | Deviation |
| 1 | D54W | 54 | D | W | 1.53 | 0.06 |
| 2 | D54Y | 54 | D | Y | 1.45 | 0.02 |
| 3 | D54F | 54 | D | F | 1.39 | 0.10 |
| 4 | D54C | 54 | D | C | 1.37 | 0.05 |
| 5 | D54K | 54 | D | K | 1.36 | 0.08 |
| 6 | D54A | 54 | D | A | 1.36 | 0.05 |
| 7 | D54R | 54 | D | R | 1.31 | 0.04 |
| 8 | D54T | 54 | D | T | 1.31 | 0.07 |
| 9 | D54V | 54 | D | V | 1.28 | 0.04 |
| 10 | D54P | 54 | D | P | 1.27 | 0.12 |
| 11 | D54L | 54 | D | L | 1.26 | 0.06 |
| 12 | D54H | 54 | D | H | 1.19 | 0.12 |
| 13 | D54M | 54 | D | M | 1.17 | 0.05 |
| 14 | D54I | 54 | D | I | 1.17 | 0.10 |
| 15 | D54S | 54 | D | S | 1.17 | 0.03 |
| 16 | D54Q | 54 | D | Q | 1.13 | 0.09 |
| 17 | D54N | 54 | D | N | 1.05 | 0.08 |
| 18 | S55N | 55 | S | N | 1.02 | 0.07 |
| 19 | F53R | 53 | F | R | 0.97 | 0.04 |
| 20 | N14A | 14 | N | A | 0.93 | 0.12 |
| 21 | V16I | 16 | V | I | 0.92 | 0.04 |
| 22 | D54G | 54 | D | G | 0.87 | 0.01 |
| 23 | S55H | 55 | S | H | 0.84 | 0.15 |
| 24 | D54E | 54 | D | E | 0.82 | 0.07 |
| 25 | F53I | 53 | F | I | 0.80 | 0.03 |
| 26 | A68D | 68 | A | D | 0.78 | 0.08 |
| 27 | S55G | 55 | S | G | 0.74 | 0.04 |
| 28 | T67D | 67 | T | D | 0.70 | 0.23 |
| 29 | N14G | 14 | N | G | 0.70 | 0.12 |
| 30 | F53Y | 53 | F | Y | 0.69 | 0.09 |
| 31 | A68W | 68 | A | W | 0.68 | 0.07 |
| 32 | F53S | 53 | F | S | 0.65 | 0.04 |
| 33 | A68N | 68 | A | N | 0.65 | 0.10 |
| 34 | S55A | 55 | S | A | 0.64 | 0.16 |
| 35 | F53A | 53 | F | A | 0.60 | 0.01 |
| 36 | F53H | 53 | F | H | 0.60 | 0.15 |
| 37 | N46L | 46 | N | L | 0.59 | 0.16 |
| 38 | V16T | 16 | V | T | 0.58 | 0.13 |
| 39 | F53T | 53 | F | T | 0.58 | 0.08 |
| 40 | A68K | 68 | A | K | 0.55 | 0.02 |
| 41 | T67C | 67 | T | C | 0.52 | 0.09 |
| 42 | A68R | 68 | A | R | 0.49 | 0.06 |
| 43 | K33G | 33 | K | G | 0.47 | 0.47 |
| 44 | F53P | 53 | F | P | 0.44 | 0.05 |
| 45 | A19V | 19 | A | V | 0.44 | 0.21 |
| 46 | L35G | 35 | L | G | 0.43 | 0.07 |
| 47 | T58H | 58 | T | H | 0.43 | 0.08 |
| 48 | S6T | 6 | S | T | 0.43 | 0.20 |
| 49 | L35P | 35 | L | P | 0.43 | 0.17 |
| 50 | I48L | 48 | I | L | 0.42 | 0.19 |
| 51 | A68L | 68 | A | L | 0.42 | 0.06 |
| 52 | K30S | 30 | K | S | 0.41 | 0.04 |
| 53 | N46K | 46 | N | K | 0.41 | 0.22 |
| 54 | R71L | 71 | R | L | 0.40 | 0.10 |
| 55 | K33S | 33 | K | S | 0.40 | 0.09 |
| 56 | F53N | 53 | F | N | 0.40 | 0.02 |
| 57 | F53V | 53 | F | V | 0.40 | 0.06 |
| 58 | I43C | 43 | I | C | 0.39 | 0.15 |
| 59 | T67Y | 67 | T | Y | 0.39 | 0.22 |
| 60 | T67M | 67 | T | M | 0.38 | 0.03 |
| 61 | L52D | 52 | L | D | 0.38 | 0.14 |
| 62 | R71W | 71 | R | W | 0.37 | 0.20 |
| 63 | F53L | 53 | F | L | 0.36 | 0.09 |
| 64 | L35T | 35 | L | T | 0.35 | 0.15 |
| 65 | N46D | 46 | N | D | 0.33 | 0.15 |
| 66 | T22V | 22 | T | V | 0.33 | 0.08 |
| 67 | K30R | 30 | K | R | 0.33 | 0.20 |
| 68 | N37A | 37 | N | A | 0.33 | 0.17 |
| 69 | L52C | 52 | L | C | 0.32 | 0.31 |
| 70 | K65M | 65 | K | M | 0.31 | 0.17 |
| 71 | F53M | 53 | F | M | 0.30 | 0.22 |
| 72 | F53G | 53 | F | G | 0.30 | 0.09 |
| 73 | K30L | 30 | K | L | 0.28 | 0.13 |
| 74 | R40P | 40 | R | P | 0.28 | 0.08 |
| 75 | T67G | 67 | T | G | 0.28 | 0.20 |
| 76 | T67I | 67 | T | I | 0.26 | 0.15 |
| 77 | S55R | 55 | S | R | 0.26 | 0.15 |
| 78 | K3S | 3S | K | S | 0.26 | 0.10 |
| 79 | L64H | 64 | L | H | 0.25 | 0.35 |
| 80 | E60H | 60 | E | H | 0.25 | 0.41 |
| 81 | A68S | 68 | A | S | 0.25 | 0.10 |
| 82 | K30W | 30 | K | W | 0.25 | 0.36 |
| 83 | T22W | 22 | T | W | 0.24 | 0.05 |
| 84 | K4P | 4P | K | p | 0.23 | 0.09 |
| 85 | S29W | 29 | S | W | 0.23 | 0.14 |
| 86 | K3Q | 3 | K | Q | 0.23 | 0.30 |
| 87 | S42I | 42 | S | I | 0.23 | 0.14 |
| 88 | L47M | 47 | L | M | 0.23 | 0.20 |
| 89 | S42V | 42 | S | V | 0.22 | 0.06 |
| 90 | K33F | 33 | K | F | 0.22 | 0.28 |
| 91 | N46T | 46 | N | T | 0.22 | 0.41 |
| 92 | K4E | 4E | K | E | 0.22 | 0.25 |
| 93 | Y72W | 72 | Y | W | 0.21 | 0.29 |
| 94 | K26Q | 26 | K | Q | 0.21 | 0.24 |
| 95 | D39K | 39 | D | K | 0.21 | 0.15 |
| 96 | T22I | 22 | T | I | 0.21 | 0.19 |
| 97 | T67S | 67 | T | S | 0.21 | 0.12 |
| 98 | N37T | 37 | N | T | 0.21 | 0.18 |
| 99 | K4Y | 4 | K | Y | 0.21 | 0.12 |
| 100 | I7Ter | 7 | I | r | 0.21 | 0.33 |
| 101 | K4A | 4A | K | A | 0.21 | 0.33 |
| 102 | K44M | 44 | K | M | 0.20 | 0.21 |
| 103 | K33Q | 33 | K | Q | 0.20 | 0.22 |
| 104 | T67W | 67 | T | W | 0.20 | 0.19 |
| 105 | T38A | 38 | T | A | 0.20 | 0.32 |
| 106 | F53K | 53 | F | K | 0.19 | 0.07 |
| 107 | F53W | 53 | F | W | 0.19 | 0.23 |
| 108 | I43M | 43 | I | M | 0.19 | 0.11 |
| 109 | L52N | 52 | L | N | 0.19 | 0.03 |
| 110 | T67Q | 67 | T | Q | 0.18 | 0.14 |
| 111 | T67N | 67 | T | N | 0.18 | 0.01 |
| 112 | N46S | 46 | N | S | 0.18 | 0.08 |
| 113 | E24M | 24 | E | M | 0.18 | 0.21 |
| 114 | K4S | 4 | K | S | 0.18 | 0.13 |
| 115 | A68Q | 68 | A | Q | 0.18 | 0.14 |
| 116 | K30T | 30 | K | T | 0.18 | 0.37 |
| 117 | T38R | 38 | T | R | 0.17 | 0.18 |
| 118 | W18F | 18 | W | F | 0.17 | 0.29 |
| 119 | K3N | 3 | K | N | 0.17 | 0.18 |
| 120 | N37Q | 37 | N | Q | 0.17 | 0.17 |
| 121 | L52W | 52 | L | W | 0.17 | 0.16 |
| 122 | L35A | 35 | L | A | 0.16 | 0.30 |
| 123 | S42M | 42 | S | M | 0.16 | 0.28 |
| 124 | K44Y | 44 | K | Y | 0.16 | 0.29 |
| 125 | L35K | 35 | L | K | 0.16 | 0.14 |
| 126 | A68G | 68 | A | G | 0.16 | 0.24 |
| 127 | T67E | 67 | T | E | 0.16 | 0.17 |
| 128 | K33P | 33 | K | P | 0.16 | 0.18 |
| 129 | K30I | 30 | K | I | 0.15 | 0.16 |
| 130 | T22M | 22 | T | M | 0.15 | 0.05 |
| 131 | K26G | 26 | K | G | 0.15 | 0.18 |
| 132 | L51A | 51 | L | A | 0.15 | 0.21 |
| 133 | G36V | 36 | G | V | 0.15 | 0.34 |
| 134 | D39N | 39 | D | N | 0.15 | 0.11 |
| 135 | I43W | 43 | I | W | 0.15 | 0.01 |
| 136 | T38E | 38 | T | E | 0.15 | 0.16 |
| 137 | T22G | 22 | T | G | 0.15 | 0.08 |
| 138 | E60S | 60 | E | S | 0.14 | 0.08 |
| 139 | M1T | 1 | M | T | 0.14 | 0.23 |
| 140 | T38M | 38 | T | M | 0.14 | 0.12 |
| 141 | A19S | 19 | A | S | 0.14 | 0.34 |
| 142 | K26T | 26 | K | T | 0.14 | 0.23 |
| 143 | K65L | 65 | K | L | 0.13 | 0.05 |
| 144 | E24P | 24 | E | P | 0.13 | 0.23 |
| 145 | K45N | 45 | K | N | 0.13 | 0.02 |
| 146 | T38W | 38 | T | W | 0.13 | 0.07 |
| 147 | K3A | 3 | K | A | 0.13 | 0.26 |
| 148 | I48F | 48 | I | F | 0.13 | 0.28 |
| 149 | I11L | 11 | I | L | 0.13 | 0.08 |
| 150 | M1I | 11 | M | I | 0.13 | 0.13 |
| 151 | E57N | 57 | E | N | 0.13 | 0.52 |
| 152 | I43V | 43 | I | V | 0.12 | 0.16 |
| 153 | T22A | 22 | T | A | 0.12 | 0.10 |
| 154 | V16M | 16 | V | M | 0.12 | 0.20 |
| 155 | K26E | 26 | K | E | 0.12 | 0.06 |
| 156 | S55C | 55 | S | C | 0.12 | 0.16 |
| 157 | N37W | 37 | N | W | 0.11 | 0.33 |
| 158 | K44C | 44 | K | C | 0.11 | 0.09 |
| 159 | T38K | 38 | T | K | 0.11 | 0.19 |
| 160 | L35M | 35 | L | M | 0.11 | 0.17 |
| 161 | K31S | 31 | K | S | 0.11 | 0.35 |
| 162 | N37S | 37 | N | S | 0.11 | 0.05 |
| 163 | K44P | 44 | K | P | 0.11 | 0.05 |
| 164 | K4Q | 4 | K | Q | 0.11 | 0.15 |
| 165 | G36D | 36 | G | D | 0.10 | 0.27 |
| 166 | K3C | 3 | K | C | 0.10 | 0.21 |
| 167 | K3M | 3 | K | M | 0.10 | 0.22 |
| 168 | T22E | 22 | T | E | 0.10 | 0.22 |
| 169 | S42F | 42 | S | F | 0.10 | 0.26 |
| 170 | S29L | 29 | S | L | 0.10 | 0.09 |
| 171 | L9S | 9 | L | S | 0.10 | 0.17 |
| 172 | N37D | 37 | N | D | 0.10 | 0.16 |
| 173 | A19C | 19 | A | C | 0.10 | 0.10 |
| 174 | K4M | 4 | K | M | 0.09 | 0.08 |
| 175 | Y72A | 72 | Y | A | 0.09 | 0.26 |
| 176 | I21Ter | 21 | I | r | 0.09 | 0.18 |
| 177 | N37H | 37 | N | H | 0.09 | 0.27 |
| 178 | L35S | 35 | L | S | 0.09 | 0.15 |
| 179 | K45H | 45 | K | H | 0.08 | 0.09 |
| 180 | VI6L | 16 | V | L | 0.08 | 0.03 |
| 181 | N46I | 46 | N | I | 0.08 | 0.11 |
| 182 | I43S | 43 | 1 | S | 0.08 | 0.19 |
| 183 | V34M | 34 | V | M | 0.08 | 0.32 |
| 184 | I43G | 43 | I | G | 0.08 | 0.34 |
| 185 | M1V | 1 | M | V | 0.08 | 0.16 |
| 186 | T38F | 38 | T | F | 0.08 | 0.19 |
| 187 | K45R | 45 | K | R | 0.08 | 0.15 |
| 188 | I11V | 11 | I | V | 0.08 | 0.08 |
| 189 | A68V | 68 | A | V | 0.08 | 0.09 |
| 190 | S29T | 29 | S | T | 0.07 | 0.20 |
| 191 | R40G | 40 | R | G | 0.07 | 0.14 |
| 192 | I43R | 43 | I | R | 0.07 | 0.15 |
| 193 | S42L | 42 | S | L | 0.07 | 0.12 |
| 194 | I43T | 43 | I | T | 0.07 | 0.10 |
| 195 | T22Y | 22 | T | Y | 0.07 | 0.29 |
| 196 | D39L | 39 | D | L | 0.07 | 0.10 |
| 197 | R40C | 40 | R | C | 0.07 | 0.09 |
| 198 | T38L | 38 | T | L | 0.07 | 0.03 |
| 199 | K26L | 26 | K | L | 0.07 | 0.49 |
| 200 | E24L | 24 | E | L | 0.06 | 0.10 |
| 201 | K33V | 33 | K | V | 0.06 | 0.33 |
| 202 | K44V | 44 | K | V | 0.06 | 0.05 |
| 203 | A68T | 68 | A | T | 0.06 | 0.06 |
| 204 | T38V | 38 | T | V | 0.06 | 0.15 |
| 205 | K3I | 3 | K | I | 0.06 | 0.35 |
| 206 | L64R | 64 | L | R | 0.06 | 0.08 |
| 207 | N37R | 37 | N | R | 0.06 | 0.12 |
| 208 | N37G | 37 | N | G | 0.06 | 0.29 |
| 209 | T38Y | 38 | T | Y | 0.06 | 0.23 |
| 210 | D39F | 39 | D | F | 0.06 | 0.08 |
| 211 | K44L | 44 | K | L | 0.05 | 0.07 |
| 212 | K26W | 26 | K | W | 0.05 | 0.04 |
| 213 | I43L | 43 | I | L | 0.05 | 0.05 |
| 214 | S42K | 42 | S | K | 0.05 | 0.08 |
| 215 | L35Q | 35 | L | Q | 0.05 | 0.12 |
| 216 | T67R | 67 | T | R | 0.05 | 0.17 |
| 217 | E57K | 57 | E | K | 0.05 | 0.03 |
| 218 | D39A | 39 | D | A | 0.04 | 0.20 |
| 219 | T62W | 62 | T | W | 0.04 | 0.19 |
| 220 | V34A | 34 | V | A | 0.04 | 0.13 |
| 221 | A61R | 61 | A | R | 0.04 | 0.20 |
| 222 | K26M | 26 | K | M | 0.04 | 0.23 |
| 223 | S29G | 29 | S | G | 0.04 | 0.23 |
| 224 | A68F | 68 | A | F | 0.04 | 0.07 |
| 225 | T22Q | 22 | T | Q | 0.04 | 0.14 |
| 226 | A68H | 68 | A | H | 0.04 | 0.32 |
| 227 | I43F | 43 | I | F | 0.04 | 0.16 |
| 228 | P28L | 28 | P | L | 0.03 | 0.18 |
| 229 | R40S | 40 | R | S | 0.03 | 0.21 |
| 230 | K3L | 3 | K | L | 0.03 | 0.47 |
| 231 | V16A | 16 | V | A | 0.03 | 0.21 |
| 232 | T38H | 38 | T | H | 0.03 | 0.20 |
| 233 | G36S | 36 | G | S | 0.03 | 0.19 |
| 234 | T22H | 22 | T | H | 0.03 | 0.11 |
| 235 | K45E | 45 | K | E | 0.03 | 0.18 |
| 236 | I43Q | 43 | I | Q | 0.03 | 0.32 |
| 237 | N37P | 37 | N | P | 0.03 | 0.05 |
| 238 | W18Y | 18 | W | Y | 0.03 | 0.23 |
| 239 | S42P | 42 | S | P | 0.03 | 0.11 |
| 240 | V34E | 34 | V | E | 0.02 | 0.11 |
| 241 | S42Y | 42 | S | Y | 0.02 | 0.12 |
| 242 | L47W | 47 | L | W | 0.02 | 0.10 |
| 243 | A68M | 68 | A | M | 0.02 | 0.27 |
| 244 | A68C | 68 | A | C | 0.02 | 0.09 |
| 245 | I43A | 43 | I | A | 0.02 | 0.11 |
| 246 | V20I | 20 | V | I | 0.02 | 0.19 |
| 247 | E57H | 57 | E | H | 0.02 | 0.45 |
| 248 | K26R | 26 | K | R | 0.02 | 0.23 |
| 249 | K65H | 65 | K | H | 0.02 | 0.11 |
| 250 | K44I | 44 | K | I | 0.02 | 0.02 |
| 251 | T22C | 22 | T | C | 0.02 | 0.15 |
| 252 | T73A | 73 | T | A | 0.02 | 0.13 |
| 253 | S42W | 42 | S | W | 0.02 | 0.21 |
| 254 | Y25W | 25 | Y | W | 0.02 | 0.05 |
| 255 | K45T | 45 | K | T | 0.01 | 0.08 |
| 256 | K65Q | 65 | K | Q | 0.01 | 0.16 |
| 257 | M1L | 1 | M | L | 0.01 | 0.06 |
| 258 | S42E | 42 | S | E | 0.01 | 0.06 |
| 259 | L64K | 64 | L | K | 0.01 | 0.12 |
| 260 | I43K | 43 | I | K | 0.01 | 0.09 |
| 261 | K26S | 26 | K | S | 0.01 | 0.31 |
| 262 | K44F | 44 | K | F | 0.01 | 0.19 |
| 263 | K4N | 4 | K | N | 0.01 | 0.32 |
| 264 | K4T | 4 | K | T | 0.01 | 0.12 |
| 265 | F32L | 32 | F | L | 0.01 | 0.10 |
| 266 | I11G | 11 | I | G | 0.01 | 0.08 |

We next evaluated the performance of novel Cas9 point mutations in the context of genome editing of human cells. We selectively introduced 9 point mutations identified in the E. coli screen on Hifi-Cas9 (R691A) (polypeptide sequence: SEQ ID NO:.4; codon-optimized nucleic acid sequences: SEQ ID NO: 5 and 6). Each mutant protein was expressed in E. coli and purified through sequential chromatography. The on-target editing efficiency of each mutant was evaluated over 3 target sites (Table 3).

TABLE 3
Target sites used to assess on-
and off-target activities of SpCas9 mutants

| Target site | Protospacer (5′-3′) | PAM |
|---|---|---|
| EMX1 | GAGTCCGAGCAGAAGAAGAA | GGG |
| HPRT 38358 | ATTTCACATAAAACTCTTTT | AGG |
| HPRT 38231 | TTTTGTAATTAACAGCTTGC | TGG |
| EMX1 | GAGTTAGAGCAGAAGAAGAA | AGG |
| off-target | | |

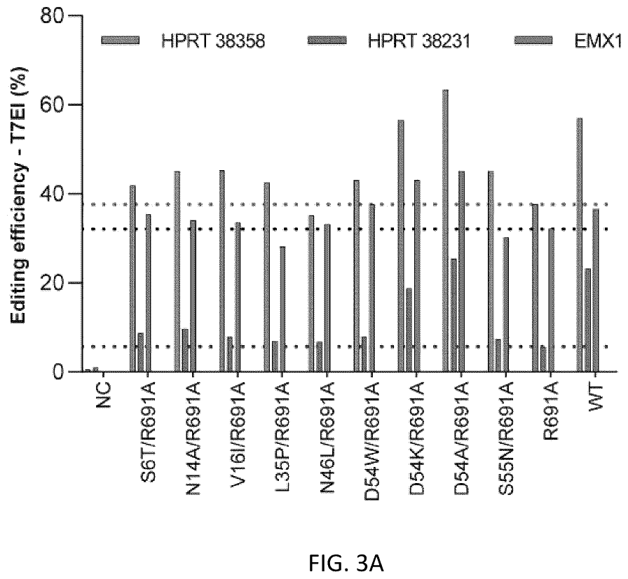

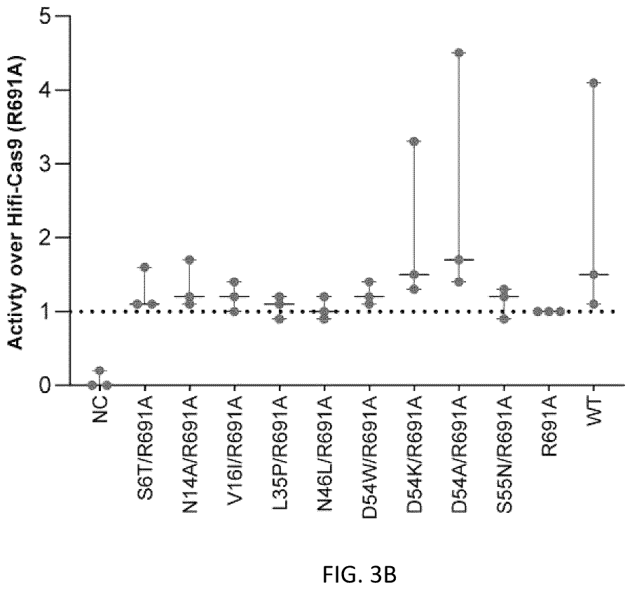

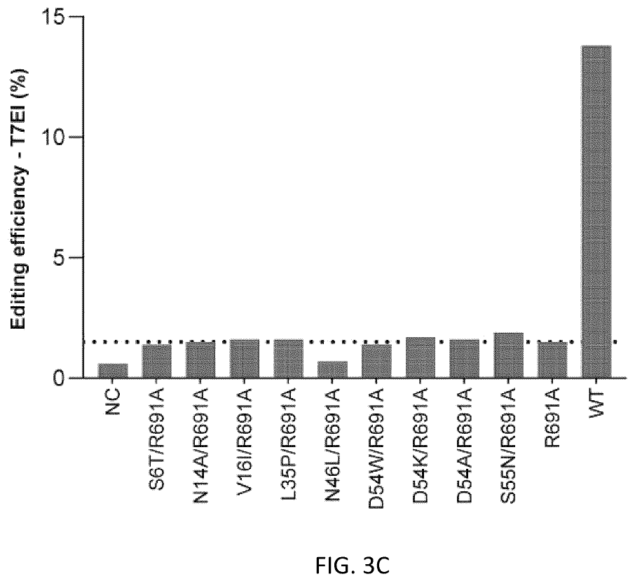

Satisfyingly, these point mutations elevated the on-target potency of Hifi-Cas9 (FIGS. 3A, B), thus validating our previous results generated in E. coli. Of note, D54A resulted in the most significant boost of on-target potency, particularly at the low-activity site (HPRT38231) that is poorly edited by Hifi-Cas9. The activity of this novel variant (D54A/R691A) is even higher than WT-SpCas9 (FIGS. 3A, B). We further studied the off-target editing level of novel Cas9 variants at EMX1 off-target site 1 (Table 3). Surprisingly, none of the mutant significantly increased the off-target effect, suggesting these novel mutants are compatible with Hifi-Cas9 (R691A), and affect the overall Cas9 protein activity at on- and off-target sites through different mechanism than R691A. In conclusion, our data demonstrated that the novel point mutations uncovered by our bacterial screen can enhance the on-target performance of Hifi-Cas9 while maintaining its excellent targeting specificity, which could serve as a direct replacement of WT-SpCas9 for human genome engineering.

Preferred single mutant Cas9 proteins include substitution mutations in the WT-Cas9 introduced at one of the positions identified in Table 2. Additional substitution mutations can be included in the amino acid backgrounds of the single mutant Cas9 protein amino acid sequences, provided that the resultant mutant Cas9 protein is active as a CRISPR/Cas endonuclease system, wherein the resultant CRISPR/Cas endonuclease system displays increased on-target editing activity relative to a wild-type CRISPR/Cas endonuclease system.

Preferred double mutant Cas9 proteins include mutations in the WT-Cas9 introduced into R691 in combination with one of the mutations presented in Table 2. Highly preferred double mutant Cas9 proteins include mutations in the WT-Cas9 introduced at having R691A in combination with one of the mutations presented in Table 2. Additional substitution mutations can be included in the amino acid backgrounds of the double mutant Cas9 protein amino acid sequences, provided that the resultant mutant Cas9 protein is active as a CRISPR/Cas endonuclease system, wherein the resultant CRISPR/Cas endonuclease system displays increased on-target editing activity relative to a wild-type CRISPR/Cas endonuclease system. Exemplary mutations presented in Table 2 may be combined, for example at other positions, such as those disclosed in U.S. patent application Ser. Nos. 15/729,491 and 15/964,041, filed Oct. 10, 2017 and Apr. 26, 2018, respectively (Attorney Docket Nos. IDT01-009-US and IDT01-009-US-CIP, respectively), the contents of which are incorporated by reference herein.

In a second aspect, an isolated ribonucleoprotein complex is provided. The RNP includes a mutant Cas9 protein and a gRNA complex. In one respect, the gRNA includes a crRNA and a tracrRNA in stoichiometric (1:1) ratio. In a second respect the crRNA includes an Alt-R® crRNA (Integrated DNA Technologies, Inc. (Coralville, IA (US)) directed against a specific editing target site for a given locus and the tracrRNA includes Alt-R® tracrRNA (Integrated DNA Technologies, Inc. (Coralville, IA (US)). In another respect the gRNA includes a sgRNA. Preferred mutant Cas9 proteins include those as described above.

In a third aspect, an isolated nucleic acid encoding a mutant Cas9 protein is provided. Preferred isolated nucleic acids encode mutant Cas9 proteins as described above. Exemplary isolated nucleic acids encoding mutant Cas9 proteins can be readily generated from a nucleic acid encoding the wild-type Cas9 protein using recombinant DNA procedures or chemical synthesis methods. Preferred nucleic acids for this purpose include those optimized for expression of the Cas9 proteins in bacteria (e.g., E. coli) or mammalian (e.g., human) cells. Exemplary codon-optimized nucleic acids for expressing WT-Cas9 (SEQ ID NO: 1) in E. coli and human cells include SEQ ID NO: 1 and 2, respectively. Exemplary codon-optimized nucleic acids for expressing mutant Cas9 protein (e.g., R691A mutant Cas9 protein; SEQ ID NO: 7) in E. coli and human cells include SEQ ID NO: 3 and 4, respectively. Moreover, the present invention contemplates fusion proteins of WT-Cas9 and mutant Cas9, wherein the coding sequences of WT-Cas9 and mutant Cas9 are fused to amino acid sequences encoding for nuclear localization (“NLS”) of the fusion protein in eukaryotic cells or amino acid sequences to facilitate purification of the proteins. Exemplary fusion proteins that include either the WT-Cas9 amino acid sequence or a mutant Cas9 amino acid sequence (e.g., R691A mutant Cas9 protein).

In one respect, the isolated nucleic acid includes mRNA encoding one of the aforementioned mutant Cas9 proteins. In a second respect, the isolated nucleic acid includes DNA encoding a gene for one of the aforementioned mutant Cas9 proteins. A preferred DNA includes a vector that encodes a gene encoding for a mutant Cas9 protein. Such delivery methods include plasmid and various viral delivery vectors as are well known to those with skill in the art. The mutant Cas9 protein can also be stably transformed into cells using suitable expression vectors to produce a cell line that constitutively or inducibly expresses the mutant Cas9. The aforementioned methods can also be applied to embryos to product progeny animals that constitutively or inducibly expresses the mutant Cas9.

In a fourth aspect, a CRISPR/Cas endonuclease system is provided. The CRISPR/Cas endonuclease system includes a mutant Cas9 protein. Preferred mutant Cas9 proteins include those as described above. In one respect, the CRISPR/Cas endonuclease system is encoded by a DNA expression vector. In one embodiment, the DNA expression vector is a plasmid-borne vector. In a second embodiment, the DNA expression vector is selected from a bacterial expression vector and a eukaryotic expression vector. In third respect, the CRISPR/Cas endonuclease system comprises a ribonucleoprotein complex comprising a mutant Cas9 protein and a gRNA complex. In one respect, the gRNA includes a crRNA and a tracrRNA in stoichiometric (1:1) ratio. In a second respect the crRNA includes an ALT-R® crRNA (Integrated DNA Technologies, Inc. (Coralville, IA (US)) directed against a specific editing target site for a given locus and the tracrRNA includes ALT-R® tracrRNA (Integrated DNA Technologies, Inc. (Coralville, IA (US)). In another respect the gRNA includes a sgRNA.

In a fifth aspect, a method of performing gene editing having increased on-target editing activity is provided. The method includes the step of contacting a candidate editing target site locus with an active CRISPR/Cas endonuclease system having a mutant Cas9 protein. In one respect, the method includes single mutant Cas9 proteins having mutations in the WT-Cas9 of SEQ ID NO: 1 introduced at one of the positions presented in Table 2. Additional substitution mutations can be included in the amino acid backgrounds of the single mutant Cas9 protein amino acid sequences, provided that the resultant mutant Cas9 protein is active as a CRISPR/Cas endonuclease system in the method, wherein the resultant CRISPR/Cas endonuclease system displays increased on-target editing activity relative to a wild-type CRISPR/Cas endonuclease system.

In another respect, the method includes Cas9 protein variants having mutations in the WT-Cas9 of SEQ ID NO: 1 introduced at positions R691 in combination with one of the mutations presented in Table 2. Additional substitution mutations can be included in the amino acid backgrounds of the double mutant Cas9 protein amino acid sequences, provided that the resultant mutant Cas9 protein is active as a CRISPR/Cas endonuclease system in the method, wherein the resultant CRISPR/Cas endonuclease system displays increased on-target editing activity relative to a wild-type CRISPR/Cas endonuclease system.

The applications of Cas9-based tools are many and varied. They include, but are not limited to: plant gene editing, yeast gene editing, mammalian gene editing, editing of cells in the organs of live animals, editing of embryos, rapid generation of knockout/knock-in animal lines, generating an animal model of disease state, correcting a disease state, inserting a reporter gene, and whole genome functional screening.

### EXAMPLE 1

### DNA and Amino Acid Sequences of Select Wild Type and Mutant Cas9 Proteins

The list below shows different wild type (WT) and mutant Cas9 nucleases described in the present invention. It will be appreciated by one with skill in the art that many different DNA sequences can encode/express the same amino acid (AA) sequence since in many cases more than one codon can encode for the same amino acid. The DNA sequences shown below only serve as example and other DNA sequences that encode the same protein (e.g., same amino acid sequence) are contemplated. It is further appreciated that additional features, elements or tags may be added to said sequences, such as NLS domains and the like. Examples are shown for WT Cas9 and mutant R691A Cas9 showing amino acid and DNA sequences for those proteins as Cas9 alone and Cas9 fused to both C-terminal and N-terminal SV40 NLS domains and a HIS-tag. For other Cas9 mutants, only the amino-acid sequences are provided, but it is contemplated that similar additions of NLS and His-tag domains may be added to facilitate use in producing recombinant proteins for use in mammalian cells. Mutations that differ from the WT sequence are identified using bold font with underline.

WT SpyCas9 AA sequence.
SEQ ID NO: 1
MDKKYSIGLDIGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGVDAKAILSARLSKSRRL
ENLIAQLPGEKKNGLFGNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQSKNGYAGYIDGGASQE
EFYKFIKPILEKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEKVLPKHSLLYEYF
TVYNELTKVKYVTEGMRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQILKEHPVENTOLONEKLYLY
YLQNGRDMYVDQELDINRLSDYDVDHIVPQSFLKDDSIDNKVLTRSDKNRGKSDNVPSEEVVKKMKNYWRQLLN
AKLITQRKFDNLTKAERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLKGSPEDNEQKQLE
VEQHKHYLDEIIEQISEFSKRVILADANLDKVLSAYNKHRDKPIREQAENIIHLFTLTNLGAPAAFKYFDTTID
RKRYTSTKEVLDATLIHQSITGLYETRIDLSQLGGD
WT SpyCas9 DNA sequence, codon optimized for expression in E. coli.
SEQ ID NO: 2
ATGGGCAGCAGCGCCCCAAAGAAGAAGCGGAAGGTCGGTATCCACGGAGTCCCAGCAGCCATGGACAAAAAGTA
CTCTATTGGCCTGGATATCGGGACCAACAGCGTCGGGTGGGCTGTTATCACCGACGAGTATAAAGTACCTTCGA
AAAAGTTCAAAGTGCTGGGCAACACCGATCGCCATTCAATCAAAAAGAACTTGATTGGTGCGCTGTTGTTTGAC
TCCGGGGAAACCGCCGAGGCGACTCGCCTTAAACGTACAGCACGTCGCCGGTACACTCGGCGTAAGAATCGCAT
TTGCTATTTGCAGGAAATCTTTAGCAACGAGATGGCAAAAGTCGATGACTCGTTTTTCCACCGCCTCGAGGAAA
GCTTTCTGGTGGAGGAAGACAAAAAGCATGAGCGTCACCCGATCTTCGGCAACATTGTCGATGAAGTAGCGTAT
CATGAAAAATACCCAACCATTTACCACTTACGCAAAAAGCTGGTGGACAGCACTGACAAAGCTGATTTGCGCCT
TATCTATTTAGCCCTGGCACATATGATTAAGTTTCGTGGTCACTTCCTGATCGAAGGAGACTTAAATCCCGACA
ACAGTGATGTTGATAAATTGTTTATTCAGCTTGTCCAAACTTACAATCAACTGTTCGAGGAAAACCCGATCAAT
GCCTCCGGTGTGGATGCAAAAGCCATTTTAAGTGCACGCCTTAGCAAGTCCCGTCGCTTAGAAAACCTTATCGC
GCAGCTGCCCGGCGAGAAAAAGAATGGTTTGTTTGGGAACCTTATTGCCTTGAGCTTAGGCCTCACCCCGAATT
TCAAAAGTAATTTCGATCTTGCAGAAGACGCCAAATTACAACTGTCGAAGGATACTTATGATGACGATCTCGAT
AATCTGTTAGCGCAGATTGGTGACCAATACGCCGATCTTTTTCTGGCGGCTAAAAATCTGAGCGACGCCATCTT
GCTTTCGGATATTCTCCGCGTTAACACCGAAATCACGAAAGCGCCTCTTAGTGCCAGCATGATTAAACGTTATG
ATGAACACCACCAGGACCTGACCTTACTCAAAGCGTTGGTTCGCCAGCAACTGCCAGAGAAGTACAAAGAAATC
TTCTTTGATCAGTCAAAGAATGGTTATGCCGGCTATATTGACGGGGGTGCAAGCCAAGAGGAATTCTACAAATT
TATCAAGCCTATTCTGGAGAAAATGGATGGCACCGAAGAGTTATTGGTGAAGCTTAACCGTGAAGACCTCCTGC
GGAAACAGCGCACATTCGATAATGGTTCGATCCCACACCAAATCCATTTGGGGGAGTTACACGCTATTTTGCGT
CGCCAGGAAGACTTTTACCCTTTCCTGAAGGATAACCGGGAGAAAATTGAGAAGATCCTTACCTTTCGTATTCC
GTATTACGTAGGCCCCTTAGCACGGGGTAATAGCCGTTTCGCGTGGATGACACGGAAGTCGGAAGAGACGATCA
CCCCGTGGAACTTCGAAGAGGTAGTCGACAAGGGCGCATCAGCGCAGTCTTTTATTGAACGTATGACGAATTTC
GATAAAAACTTGCCCAATGAGAAGGTGCTTCCGAAACATTCCTTGTTATATGAATATTTTACAGTTTACAACGA
GCTGACCAAGGTTAAATACGTGACGGAAGGAATGCGCAAGCCCGCTTTTCTTAGCGGTGAGCAAAAAAAGGCGA
TCGTCGACCTGTTATTCAAAACGAATCGTAAGGTGACTGTAAAGCAACTCAAAGAAGATTACTTCAAAAAGATT
GAGTGCTTCGACAGCGTCGAAATCTCTGGGGTAGAGGATCGGTTTAACGCAAGTTTAGGTACCTACCATGACCT
GCTTAAAATCATTAAGGATAAAGACTTCTTAGATAATGAAGAGAACGAAGATATTCTCGAGGACATCGTCTTGA
CGTTAACCTTATTTGAGGATCGTGAAATGATTGAGGAACGCCTCAAAACTTATGCCCACCTGTTCGACGATAAG
GTGATGAAGCAGCTGAAACGTCGGCGCTACACAGGATGGGGCCGCTTGAGTCGCAAACTTATTAACGGAATCCG
TGACAAGCAATCCGGCAAAACGATTCTGGATTTCTTGAAGTCGGACGGATTTGCTAATCGCAACTTCATGCAGT
TGATCCATGATGACTCCCTGACTTTTAAAGAGGATATTCAAAAGGCGCAGGTTAGTGGTCAAGGCGACAGCTTA
CACGAACACATCGCAAATTTGGCTGGTTCGCCGGCCATTAAAAAGGGGATCCTCCAGACCGTGAAAGTTGTAGA
TGAGCTTGTTAAGGTCATGGGTCGTCATAAGCCCGAAAACATCGTGATTGAAATGGCGCGGGAGAATCAAACGA
CCCAGAAAGGACAAAAGAATAGCCGTGAACGGATGAAGCGGATCGAGGAAGGCATTAAAGAGCTGGGGTCTCAA
ATCTTGAAGGAACACCCTGTGGAGAACACTCAGCTCCAAAATGAAAAACTTTACCTGTACTATTTGCAGAACGG
ACGCGATATGTACGTGGACCAAGAGTTGGATATTAATCGGCTGAGTGACTACGACGTTGATCATATCGTCCCGC
AGAGCTTCCTCAAAGACGATTCTATTGACAATAAGGTACTGACGCGCTCTGATAAAAACCGTGGTAAGTCGGAC
AACGTGCCCTCCGAAGAGGTTGTGAAAAAGATGAAAAATTATTGGCGCCAGCTTTTAAACGCGAAGCTGATCAC
ACAACGTAAATTCGATAATTTGACCAAGGCTGAACGGGGTGGCCTGAGCGAGTTAGATAAGGCAGGATTTATTA
AACGCCAGTTAGTGGAGACTCGTCAAATCACCAAACATGTCGCGCAGATTTTGGACAGCCGGATGAACACCAAG
TAGGATGAAAATGACAAACTGATCCGTGAGGTGAAAGTCATTACTCTGAAGTCCAAATTAGTTAGTGATTTCCG
GAAGGACTTTCAATTCTACAAAGTCCGTGAAATTAATAACTATCATCACGCACATGACGCGTACCTGAATGCAG
TGGTTGGGACCGCCCTTATCAAGAAATATCCTAAGCTGGAGTCGGAGTTTGTCTATGGCGACTATAAGGTATAC
GATGTTCGCAAAATGATTGCGAAATCTGAGCAGGAGATCGGTAAGGCAACCGCAAAATATTTCTTTTACTCAAA
CATTATGAATTTCTTTAAGACAGAAATCACTCTGGCCAACGGGGAGATTCGCAAACGTCCGTTGATCGAAACAA
ACGGCGAGACTGGCGAAATTGTTTGGGACAAAGGGCGTGATTTCGCGACGGTGCGCAAGGTACTGAGCATGCCT
CAAGTCAATATTGTTAAGAAAACCGAAGTGCAGACGGGCGGGTTTTCCAAGGAAAGCATCTTACCCAAACGTAA
TTCAGATAAACTTATTGCACGCAAAAAGGACTGGGATCCGAAAAAGTATGGAGGCTTCGACAGTCCAACCGTAG
CCTACTCTGTTCTCGTTGTAGCGAAAGTAGAAAAGGGTAAATCCAAGAAACTGAAATCTGTCAAGGAGTTGCTT
GGAATCACCATTATGGAGCGTAGCTCCTTCGAGAAGAACCCGATTGACTTTCTGGAAGCCAAAGGATATAAAGA
GGTCAAGAAAGATCTTATCATTAAGCTGCCTAAGTATTCACTCTTCGAGCTGGAAAATGGTCGTAAACGCATGC
TCGCTTCTGCCGGCGAGTTGCAGAAGGGCAATGAATTAGCACTTCCATCAAAGTACGTTAACTTCCTGTATTTG
GCCAGCCATTACGAGAAACTGAAGGGGTCTCCAGAGGACAACGAACAGAAACAATTATTTGTAGAGCAGCACAA
GCATTATCTTGATGAAATCATTGAGCAAATTTCCGAATTCAGTAAACGCGTAATCCTGGCCGATGCAAACCTCG
ACAAGGTGCTGAGCGCTTACAATAAGCATCGCGACAAACCTATCCGTGAGCAGGCTGAAAATATCATTCACCTG
TTCACATTAACGAACCTGGGCGCTCCGGCCGCTTTTAAATATTTCGACACGACAATCGACCGTAAGCGCTATAC
CAGTACGAAAGAAGTGTTGGATGCGACCCTTATTCACCAGTCAATTACAGGATTATATGAGACCCGTATCGACC
TTAGCCAATTAGGTGGGGATGCGGCCCCGAAGAAAAAACGCAAAGTGGATCCGAAGAAAAAACGCAAAGTGGCG
GCCGCACTCGAGCACCACCACCACCACCACTGA
WT SpyCas9 DNA sequence, codon optimized for expression in H. sapiens.
SEQ ID NO: 3
ATGGGCAAGCCCATCCCTAACCCCCTGTTGGGGCTGGACAGCACCGCTCCCAAAAAGAAAAGGAAGGTGGGCAT
TCACGGCGTGCCTGCGGCCGACAAAAAGTACAGCATCGGCCTTGATATCGGCACCAATAGCGTGGGCTGGGCCG
TTATCACAGACGAATACAAGGTACCCAGCAAGAAGTTCAAGGTGCTGGGGAATACAGACAGGCACTCTATCAAG
AAAAACCTTATCGGGGCTCTGCTGTTTGACTCAGGCGAGACCGCCGAGGCCACCAGGTTGAAGAGGACCGCAAG
GCGAAGGTACACCCGGAGGAAGAACAGGATCTGCTATCTGCAGGAGATCTTCAGCAACGAGATGGCCAAGGTGG
ACGACAGCTTCTTCCACAGGCTGGAGGAGAGCTTCCTTGTCGAGGAGGATAAGAAGCACGAACGACACCCCATC
TTCGGCAACATAGTCGACGAGGTCGCTTATCACGAGAAGTACCCCACCATCTACCACCTGCGAAAGAAATTGGT
GGATAGCACCGATAAAGCCGACTTGCGACTTATCTACTTGGCTCTGGCGCACATGATTAAGTTCAGGGGCCACT
TCCTGATCGAGGGCGACCTTAACCCCGACAACAGTGACGTAGACAAATTGTTCATCCAGCTTGTACAGACCTAT
AACCAGCTGTTCGAGGAAAACCCTATTAACGCCAGCGGGGTGGATGCGAAGGCCATACTTAGCGCCAGGCTGAG
CAAAAGCAGGCGCTTGGAGAACCTGATAGCCCAGCTGCCCGGTGAAAAGAAGAACGGCCTCTTCGGTAATCTGA
TTGCCCTGAGCCTGGGCCTGACCCCCAACTTCAAGAGCAACTTCGACCTGGCAGAAGATGCCAAGCTGCAGTTG
AGTAAGGACACCTATGACGACGACTTGGACAATCTGCTCGCCCAAATCGGCGACCAGTACGCTGACCTGTTCCT
CGCCGCCAAGAACCTTTCTGACGCAATCCTGCTTAGCGATATCCTTAGGGTGAACACAGAGATCACCAAGGCCC
CCCTGAGCGCCAGCATGATCAAGAGGTACGACGAGCACCATCAGGACCTGACCCTTCTGAAGGCCCTGGTGAGG
CAGCAACTGCCCGAGAAGTACAAGGAGATCTTTTTCGACCAGAGCAAGAACGGCTACGCCGGCTACATCGACGG
CGGAGCCAGCCAAGAGGAGTTCTACAAGTTCATCAAGCCCATCCTGGAGAAGATGGATGGCACCGAGGAGCTGC
TGGTGAAGCTGAACAGGGAAGATTTGCTCCGGAAGCAGAGGACCTTTGACAACGGTAGCATCCCCCACCAGATC
CACCTGGGCGAGCTGCACGCAATACTGAGGCGACAGGAGGATTTCTACCCCTTCCTCAAGGACAATAGGGAGAA
AATCGAAAAGATTCTGACCTTCAGGATCCCCTACTACGTGGGCCCTCTTGCCAGGGGCAACAGCCGATTCGCTT
GGATGACAAGAAAGAGCGAGGAGACCATCACCCCCTGGAACTTCGAGGAAGTGGTGGACAAAGGAGCAAGCGCG
CAGTCTTTCATCGAACGGATGACCAATTTCGACAAAAACCTGCCTAACGAGAAGGTGCTGCCCAAGCACAGCCT
GCTTTACGAGTACTTCACCGTGTACAACGAGCTCACCAAGGTGAAATATGTGACCGAGGGCATGCGAAAACCCG
CTTTCCTGAGCGGCGAGCAGAAGAAGGCCATCGTGGACCTGCTGTTCAAGACCAACAGGAAGGTGACCGTGAAG
CAGCTGAAGGAGGACTACTTCAAGAAGATCGAGTGCTTTGATAGCGTGGAAATAAGCGGCGTGGAGGACAGGTT
CAACGCCAGCCTGGGCACCTACCACGACTTGTTGAAGATAATCAAAGACAAGGATTTCCTGGATAATGAGGAGA
ACGAGGATATACTCGAGGACATCGTGCTGACTTTGACCCTGTTTGAGGACCGAGAGATGATTGAAGAAAGGCTC
AAAACCTACGCCCACCTGTTCGACGACAAAGTGATGAAACAACTGAAGAGACGAAGATACACCGGCTGGGGCAG
ACTGTCCAGGAAGCTCATCAACGGCATTAGGGACAAGCAGAGCGGCAAGACCATCCTGGATTTCCTGAAGTCCG
ACGGCTTCGCCAACCGAAACTTCATGCAGCTGATTCACGATGACAGCTTGACCTTCAAGGAGGACATCCAGAAG
GCCCAGGTTAGCGGCCAGGGCGACTCCCTGCACGAACATATTGCAAACCTGGCAGGCTCCCCTGCGATCAAGAA
GGGCATACTGCAGACCGTTAAGGTTGTGGACGAATTGGTCAAGGTCATGGGCAGGCACAAGCCCGAAAACATAG
TTATAGAGATGGCCAGAGAGAACCAGACCACCCAAAAGGGCCAGAAGAACAGCCGGGAGCGCATGAAAAGGATC
GAGGAGGGTATCAAGGAACTCGGAAGCCAGATCCTCAAAGAGCACCCCGTGGAGAATACCCAGCTCCAGAACGA
GAAGCTGTACCTGTACTACCTGCAGAACGGCAGGGACATGTACGTTGACCAGGAGTTGGACATCAACAGGCTTT
CAGACTATGACGTGGATCACATAGTGCCCCAGAGCTTTCTTAAAGACGATAGCATCGACAACAAGGTCCTGACC
CGCTCCGACAAAAACAGGGGCAAAAGCGACAACGTGCCAAGCGAAGAGGTGGTTAAAAAGATGAAGAACTACTG
GAGGCAACTGCTCAACGCGAAATTGATCACCCAGAGAAAGTTCGATAACCTGACCAAGGCCGAGAGGGGCGGAC
TCTCCGAACTTGACAAAGCGGGCTTCATAAAGAGGCAGCTGGTCGAGACCCGACAGATCACGAAGCACGTGGCC
CAAATCCTCGACAGCAGAATGAATACCAAGTACGATGAGAATGACAAACTCATCAGGGAAGTGAAAGTGATTAC
CCTGAAGAGCAAGTTGGTGTCCGACTTTCGCAAAGATTTCCAGTTCTACAAGGTGAGGGAGATCAACAACTACC
ACCATGCCCACGACGCATACCTGAACGCCGTGGTCGGCACCGCCCTGATTAAGAAGTATCCAAAGCTGGAGTCC
GAATTTGTCTACGGCGACTACAAAGTTTACGATGTGAGGAAGATGATCGCTAAGAGCGAACAGGAGATCGGCAA
GGCCACCGCTAAGTATTTCTTCTACAGCAACATCATGAACTTTTTCAAGACCGAGATCACACTTGCCAACGGCG
AAATCAGGAAGAGGCCGCTTATCGAGACCAACGGTGAGACCGGCGAGATCGTGTGGGACAAGGGCAGGGACTTC
GCCACCGTGAGGAAAGTCCTGAGCATGCCCCAGGTGAATATTGTGAAAAAAACTGAGGTGCAGACAGGCGGCTT
TAGCAAGGAATCCATCCTGCCCAAGAGGAACAGCGACAAGCTGATCGCCCGGAAGAAGGACTGGGACCCTAAGA
AGTATGGAGGCTTCGACAGCCCCACCGTAGCCTACAGCGTGCTGGTGGTCGCGAAGGTAGAGAAGGGGAAGAGC
AAGAAACTGAAGAGCGTGAAGGAGCTGCTCGGCATAACCATCATGGAGAGGTCCAGCTTTGAGAAGAACCCCAT
TGACTTTTTGGAAGCCAAGGGCTACAAAGAGGTCAAAAAGGACCTGATCATCAAACTCCCCAAGTACTCCCTGT
TTGAATTGGAGAACGGCAGAAAGAGGATGCTGGCGAGCGCTGGGGAACTGCAAAAGGGCAACGAACTGGCGCTG
CCCAGCAAGTACGTGAATTTTCTGTACCTGGCGTCCCACTACGAAAAGCTGAAAGGCAGCCCCGAGGACAACGA
GCAGAAGCAGCTGTTCGTGGAGCAGCACAAGCATTACCTGGACGAGATAATCGAGCAAATCAGCGAGTTCAGCA
AGAGGGTGATTCTGGCCGACGCGAACCTGGATAAGGTCCTCAGCGCCTACAACAAGCACCGAGACAAACCCATC
AGGGAGCAGGCCGAGAATATCATACACCTGTTCACCCTGACAAATCTGGGCGCACCTGCGGCATTCAAATACTT
CGATACCACCATCGACAGGAAAAGGTACACTAGCACTAAGGAGGTGCTGGATGCCACCTTGATCCACCAGTCCA
TTACCGGCCTGTATGAGACCAGGATCGACCTGAGCCAGCTTGGAGGCGACTCTAGGGCGGACCCAAAAAAGAAA
AGGAAGGTGGAATTCCACCACACTGGACTAGTGGATCCGAGCTCGGTACCAAGCTTAAGTTTAAACCGCTGA
R691A mutant SpyCas9 AA sequence.
SEQ ID NO: 4
MDKKYSIGLDIGTNSVGWAVITDEYKVPSKKFKVLGNTDRHSIKKNLIGALLFDSGETAEATRLKRTARRRYTR
RKNRICYLQEIFSNEMAKVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGVDAKAILSARLSKSRRL
ENLIAQLPGEKKNGLFGNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQSKNGYAGYIDGGASQE
EFYKFIKPILEKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEKVLPKHSLLYEYF
TVYNELTKVKYVTEGMRKPAFLSGEQKKAIVDLLFKTNRKVTVKQLKEDYFKKIECFDSVEISGVEDRFNASLG
TYHDLLKIIKDKDFLDNEENEDILEDIVLTLTLFEDREMIEERLKTYAHLFDDKVMKQLKRRRYTGWGRLSRKL
INGIRDKQSGKTILDFLKSDGFANANFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNRGKSDNVPSEEVVKKMKNYWRQLLN
AKLITQRKFDNLTKAERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTNLGAPAAFKYFDTTID
RKRYTSTKEVLDATLIHQSITGLYETRIDLSQLGGD
R691A mutant SpyCas9 DNA sequence, codon optimized for expression in E. coli.
SEQ ID NO: 5
ATGGGCAGCAGCGCCCCAAAGAAGAAGCGGAAGGTCGGTATCCACGGAGTCCCAGCAGCCATGGACAAAAAGTA
CTCTATTGGCCTGGATATCGGGACCAACAGCGTCGGGTGGGCTGTTATCACCGACGAGTATAAAGTACCTTCGA
AAAAGTTCAAAGTGCTGGGCAACACCGATCGCCATTCAATCAAAAAGAACTTGATTGGTGCGCTGTTGTTTGAC
TCCGGGGAAACCGCCGAGGCGACTCGCCTTAAACGTACAGCACGTCGCCGGTACACTCGGCGTAAGAATCGCAT
TTGCTATTTGCAGGAAATCTTTAGCAACGAGATGGCAAAAGTCGATGACTCGTTTTTCCACCGCCTCGAGGAAA
GCTTTCTGGTGGAGGAAGACAAAAAGCATGAGCGTCACCCGATCTTCGGCAACATTGTCGATGAAGTAGCGTAT
CATGAAAAATACCCAACCATTTACCACTTACGCAAAAAGCTGGTGGACAGCACTGACAAAGCTGATTTGCGCCT
TATCTATTTAGCCCTGGCACATATGATTAAGTTTCGTGGTCACTTCCTGATCGAAGGAGACTTAAATCCCGACA
ACAGTGATGTTGATAAATTGTTTATTCAGCTTGTCCAAACTTACAATCAACTGTTCGAGGAAAACCCGATCAAT
GCCTCCGGTGTGGATGCAAAAGCCATTTTAAGTGCACGCCTTAGCAAGTCCCGTCGCTTAGAAAACCTTATCGC
GCAGCTGCCCGGCGAGAAAAAGAATGGTTTGTTTGGGAACCTTATTGCCTTGAGCTTAGGCCTCACCCCGAATT
TCAAAAGTAATTTCGATCTTGCAGAAGACGCCAAATTACAACTGTCGAAGGATACTTATGATGACGATCTCGAT
AATCTGTTAGCGCAGATTGGTGACCAATACGCCGATCTTTTTCTGGCGGCTAAAAATCTGAGCGACGCCATCTT
GCTTTCGGATATTCTCCGCGTTAACACCGAAATCACGAAAGCGCCTCTTAGTGCCAGCATGATTAAACGTTATG
ATGAACACCACCAGGACCTGACCTTACTCAAAGCGTTGGTTCGCCAGCAACTGCCAGAGAAGTACAAAGAAATC
TTCTTTGATCAGTCAAAGAATGGTTATGCCGGCTATATTGACGGGGGTGCAAGCCAAGAGGAATTCTACAAATT
TATCAAGCCTATTCTGGAGAAAATGGATGGCACCGAAGAGTTATTGGTGAAGCTTAACCGTGAAGACCTCCTGC
GGAAACAGCGCACATTCGATAATGGTTCGATCCCACACCAAATCCATTTGGGGGAGTTACACGCTATTTTGCGT
CGCCAGGAAGACTTTTACCCTTTCCTGAAGGATAACCGGGAGAAAATTGAGAAGATCCTTACCTTTCGTATTCC
GTATTACGTAGGCCCCTTAGCACGGGGTAATAGCCGTTTCGCGTGGATGACACGGAAGTCGGAAGAGACGATCA
CCCCGTGGAACTTCGAAGAGGTAGTCGACAAGGGCGCATCAGCGCAGTCTTTTATTGAACGTATGACGAATTTC
GATAAAAACTTGCCCAATGAGAAGGTGCTTCCGAAACATTCCTTGTTATATGAATATTTTACAGTTTACAACGA
GCTGACCAAGGTTAAATACGTGACGGAAGGAATGCGCAAGCCCGCTTTTCTTAGCGGTGAGCAAAAAAAGGCGA
TCGTCGACCTGTTATTCAAAACGAATCGTAAGGTGACTGTAAAGCAACTCAAAGAAGATTACTTCAAAAAGATT
GAGTGCTTCGACAGCGTCGAAATCTCTGGGGTAGAGGATCGGTTTAACGCAAGTTTAGGTACCTACCATGACCT
GCTTAAAATCATTAAGGATAAAGACTTCTTAGATAATGAAGAGAACGAAGATATTCTCGAGGACATCGTCTTGA
CGTTAACCTTATTTGAGGATCGTGAAATGATTGAGGAACGCCTCAAAACTTATGCCCACCTGTTCGACGATAAG
GTGATGAAGCAGCTGAAACGTCGGCGCTACACAGGATGGGGCCGCTTGAGTCGCAAACTTATTAACGGAATCCG
TGACAAGCAATCCGGCAAAACGATTCTGGATTTCTTGAAGTCGGACGGATTTGCTAATGCGAACTTCATGCAGT
TGATCCATGATGACTCCCTGACTTTTAAAGAGGATATTCAAAAGGCGCAGGTTAGTGGTCAAGGCGACAGCTTA
CACGAACACATCGCAAATTTGGCTGGTTCGCCGGCCATTAAAAAGGGGATCCTCCAGACCGTGAAAGTTGTAGA
TGAGCTTGTTAAGGTCATGGGTCGTCATAAGCCCGAAAACATCGTGATTGAAATGGCGCGGGAGAATCAAACGA
CCCAGAAAGGACAAAAGAATAGCCGTGAACGGATGAAGCGGATCGAGGAAGGCATTAAAGAGCTGGGGTCTCAA
ATCTTGAAGGAACACCCTGTGGAGAACACTCAGCTCCAAAATGAAAAACTTTACCTGTACTATTTGCAGAACGG
ACGCGATATGTACGTGGACCAAGAGTTGGATATTAATCGGCTGAGTGACTACGACGTTGATCATATCGTCCCGC
AGAGCTTCCTCAAAGACGATTCTATTGACAATAAGGTACTGACGCGCTCTGATAAAAACCGTGGTAAGTCGGAC
AACGTGCCCTCCGAAGAGGTTGTGAAAAAGATGAAAAATTATTGGCGCCAGCTTTTAAACGCGAAGCTGATCAC
ACAACGTAAATTCGATAATTTGACCAAGGCTGAACGGGGTGGCCTGAGCGAGTTAGATAAGGCAGGATTTATTA
AACGCCAGTTAGTGGAGACTCGTCAAATCACCAAACATGTCGCGCAGATTTTGGACAGCCGGATGAACACCAAG
TAGGATGAAAATGACAAACTGATCCGTGAGGTGAAAGTCATTACTCTGAAGTCCAAATTAGTTAGTGATTTCCG
GAAGGACTTTCAATTCTACAAAGTCCGTGAAATTAATAACTATCATCACGCACATGACGCGTACCTGAATGCAG
TGGTTGGGACCGCCCTTATCAAGAAATATCCTAAGCTGGAGTCGGAGTTTGTCTATGGCGACTATAAGGTATAC
GATGTTCGCAAAATGATTGCGAAATCTGAGCAGGAGATCGGTAAGGCAACCGCAAAATATTTCTTTTACTCAAA
CATTATGAATTTCTTTAAGACAGAAATCACTCTGGCCAACGGGGAGATTCGCAAACGTCCGTTGATCGAAACAA
ACGGCGAGACTGGCGAAATTGTTTGGGACAAAGGGCGTGATTTCGCGACGGTGCGCAAGGTACTGAGCATGCCT
CAAGTCAATATTGTTAAGAAAACCGAAGTGCAGACGGGCGGGTTTTCCAAGGAAAGCATCTTACCCAAACGTAA
TTCAGATAAACTTATTGCACGCAAAAAGGACTGGGATCCGAAAAAGTATGGAGGCTTCGACAGTCCAACCGTAG
CCTACTCTGTTCTCGTTGTAGCGAAAGTAGAAAAGGGTAAATCCAAGAAACTGAAATCTGTCAAGGAGTTGCTT
GGAATCACCATTATGGAGCGTAGCTCCTTCGAGAAGAACCCGATTGACTTTCTGGAAGCCAAAGGATATAAAGA
GGTCAAGAAAGATCTTATCATTAAGCTGCCTAAGTATTCACTCTTCGAGCTGGAAAATGGTCGTAAACGCATGC
TCGCTTCTGCCGGCGAGTTGCAGAAGGGCAATGAATTAGCACTTCCATCAAAGTACGTTAACTTCCTGTATTTG
GCCAGCCATTACGAGAAACTGAAGGGGTCTCCAGAGGACAACGAACAGAAACAATTATTTGTAGAGCAGCACAA
GCATTATCTTGATGAAATCATTGAGCAAATTTCCGAATTCAGTAAACGCGTAATCCTGGCCGATGCAAACCTCG
ACAAGGTGCTGAGCGCTTACAATAAGCATCGCGACAAACCTATCCGTGAGCAGGCTGAAAATATCATTCACCTG
TTCACATTAACGAACCTGGGCGCTCCGGCCGCTTTTAAATATTTCGACACGACAATCGACCGTAAGCGCTATAC
CAGTACGAAAGAAGTGTTGGATGCGACCCTTATTCACCAGTCAATTACAGGATTATATGAGACCCGTATCGACC
TTAGCCAATTAGGTGGGGATGCGGCCCCGAAGAAAAAACGCAAAGTGGATCCGAAGAAAAAACGCAAAGTGGCG
GCCGCACTCGAGCACCACCACCACCACCACTGA
R691A mutant SpyCas9 DNA sequence, codon optimized for expression in H. sapiens.
SEQ ID NO: 6
ATGGGCAAGCCCATCCCTAACCCCCTGTTGGGGCTGGACAGCACCGCTCCCAAAAAGAAAAGGAAGGTGGGCAT
TCACGGCGTGCCTGCGGCCGACAAAAAGTACAGCATCGGCCTTGATATCGGCACCAATAGCGTGGGCTGGGCCG
TTATCACAGACGAATACAAGGTACCCAGCAAGAAGTTCAAGGTGCTGGGGAATACAGACAGGCACTCTATCAAG
AAAAACCTTATCGGGGCTCTGCTGTTTGACTCAGGCGAGACCGCCGAGGCCACCAGGTTGAAGAGGACCGCAAG
GCGAAGGTACACCCGGAGGAAGAACAGGATCTGCTATCTGCAGGAGATCTTCAGCAACGAGATGGCCAAGGTGG
ACGACAGCTTCTTCCACAGGCTGGAGGAGAGCTTCCTTGTCGAGGAGGATAAGAAGCACGAACGACACCCCATC
TTCGGCAACATAGTCGACGAGGTCGCTTATCACGAGAAGTACCCCACCATCTACCACCTGCGAAAGAAATTGGT
GGATAGCACCGATAAAGCCGACTTGCGACTTATCTACTTGGCTCTGGCGCACATGATTAAGTTCAGGGGCCACT
TCCTGATCGAGGGCGACCTTAACCCCGACAACAGTGACGTAGACAAATTGTTCATCCAGCTTGTACAGACCTAT
AACCAGCTGTTCGAGGAAAACCCTATTAACGCCAGCGGGGTGGATGCGAAGGCCATACTTAGCGCCAGGCTGAG
CAAAAGCAGGCGCTTGGAGAACCTGATAGCCCAGCTGCCCGGTGAAAAGAAGAACGGCCTCTTCGGTAATCTGA
TTGCCCTGAGCCTGGGCCTGACCCCCAACTTCAAGAGCAACTTCGACCTGGCAGAAGATGCCAAGCTGCAGTTG
AGTAAGGACACCTATGACGACGACTTGGACAATCTGCTCGCCCAAATCGGCGACCAGTACGCTGACCTGTTCCT
CGCCGCCAAGAACCTTTCTGACGCAATCCTGCTTAGCGATATCCTTAGGGTGAACACAGAGATCACCAAGGCCC
CCCTGAGCGCCAGCATGATCAAGAGGTACGACGAGCACCATCAGGACCTGACCCTTCTGAAGGCCCTGGTGAGG
CAGCAACTGCCCGAGAAGTACAAGGAGATCTTTTTCGACCAGAGCAAGAACGGCTACGCCGGCTACATCGACGG
CGGAGCCAGCCAAGAGGAGTTCTACAAGTTCATCAAGCCCATCCTGGAGAAGATGGATGGCACCGAGGAGCTGC
TGGTGAAGCTGAACAGGGAAGATTTGCTCCGGAAGCAGAGGACCTTTGACAACGGTAGCATCCCCCACCAGATC
CACCTGGGCGAGCTGCACGCAATACTGAGGCGACAGGAGGATTTCTACCCCTTCCTCAAGGACAATAGGGAGAA
AATCGAAAAGATTCTGACCTTCAGGATCCCCTACTACGTGGGCCCTCTTGCCAGGGGCAACAGCCGATTCGCTT
GGATGACAAGAAAGAGCGAGGAGACCATCACCCCCTGGAACTTCGAGGAAGTGGTGGACAAAGGAGCAAGCGCG
CAGTCTTTCATCGAACGGATGACCAATTTCGACAAAAACCTGCCTAACGAGAAGGTGCTGCCCAAGCACAGCCT
GCTTTACGAGTACTTCACCGTGTACAACGAGCTCACCAAGGTGAAATATGTGACCGAGGGCATGCGAAAACCCG
CTTTCCTGAGCGGCGAGCAGAAGAAGGCCATCGTGGACCTGCTGTTCAAGACCAACAGGAAGGTGACCGTGAAG
CAGCTGAAGGAGGACTACTTCAAGAAGATCGAGTGCTTTGATAGCGTGGAAATAAGCGGCGTGGAGGACAGGTT
CAACGCCAGCCTGGGCACCTACCACGACTTGTTGAAGATAATCAAAGACAAGGATTTCCTGGATAATGAGGAGA
ACGAGGATATACTCGAGGACATCGTGCTGACTTTGACCCTGTTTGAGGACCGAGAGATGATTGAAGAAAGGCTC
AAAACCTACGCCCACCTGTTCGACGACAAAGTGATGAAACAACTGAAGAGACGAAGATACACCGGCTGGGGCAG
ACTGTCCAGGAAGCTCATCAACGGCATTAGGGACAAGCAGAGCGGCAAGACCATCCTGGATTTCCTGAAGTCCG
ACGGCTTCGCCAACGCCAACTTCATGCAGCTGATTCACGATGACAGCTTGACCTTCAAGGAGGACATCCAGAAG
GCCCAGGTTAGCGGCCAGGGCGACTCCCTGCACGAACATATTGCAAACCTGGCAGGCTCCCCTGCGATCAAGAA
GGGCATACTGCAGACCGTTAAGGTTGTGGACGAATTGGTCAAGGTCATGGGCAGGCACAAGCCCGAAAACATAG
TTATAGAGATGGCCAGAGAGAACCAGACCACCCAAAAGGGCCAGAAGAACAGCCGGGAGCGCATGAAAAGGATC
GAGGAGGGTATCAAGGAACTCGGAAGCCAGATCCTCAAAGAGCACCCCGTGGAGAATACCCAGCTCCAGAACGA
GAAGCTGTACCTGTACTACCTGCAGAACGGCAGGGACATGTACGTTGACCAGGAGTTGGACATCAACAGGCTTT
CAGACTATGACGTGGATCACATAGTGCCCCAGAGCTTTCTTAAAGACGATAGCATCGACAACAAGGTCCTGACC
CGCTCCGACAAAAACAGGGGCAAAAGCGACAACGTGCCAAGCGAAGAGGTGGTTAAAAAGATGAAGAACTACTG
GAGGCAACTGCTCAACGCGAAATTGATCACCCAGAGAAAGTTCGATAACCTGACCAAGGCCGAGAGGGGCGGAC
TCTCCGAACTTGACAAAGCGGGCTTCATAAAGAGGCAGCTGGTCGAGACCCGACAGATCACGAAGCACGTGGCC
CAAATCCTCGACAGCAGAATGAATACCAAGTACGATGAGAATGACAAACTCATCAGGGAAGTGAAAGTGATTAC
CCTGAAGAGCAAGTTGGTGTCCGACTTTCGCAAAGATTTCCAGTTCTACAAGGTGAGGGAGATCAACAACTACC
ACCATGCCCACGACGCATACCTGAACGCCGTGGTCGGCACCGCCCTGATTAAGAAGTATCCAAAGCTGGAGTCC
GAATTTGTCTACGGCGACTACAAAGTTTACGATGTGAGGAAGATGATCGCTAAGAGCGAACAGGAGATCGGCAA
GGCCACCGCTAAGTATTTCTTCTACAGCAACATCATGAACTTTTTCAAGACCGAGATCACACTTGCCAACGGCG
AAATCAGGAAGAGGCCGCTTATCGAGACCAACGGTGAGACCGGCGAGATCGTGTGGGACAAGGGCAGGGACTTC
GCCACCGTGAGGAAAGTCCTGAGCATGCCCCAGGTGAATATTGTGAAAAAAACTGAGGTGCAGACAGGCGGCTT
TAGCAAGGAATCCATCCTGCCCAAGAGGAACAGCGACAAGCTGATCGCCCGGAAGAAGGACTGGGACCCTAAGA
AGTATGGAGGCTTCGACAGCCCCACCGTAGCCTACAGCGTGCTGGTGGTCGCGAAGGTAGAGAAGGGGAAGAGC
AAGAAACTGAAGAGCGTGAAGGAGCTGCTCGGCATAACCATCATGGAGAGGTCCAGCTTTGAGAAGAACCCCAT
TGACTTTTTGGAAGCCAAGGGCTACAAAGAGGTCAAAAAGGACCTGATCATCAAACTCCCCAAGTACTCCCTGT
TTGAATTGGAGAACGGCAGAAAGAGGATGCTGGCGAGCGCTGGGGAACTGCAAAAGGGCAACGAACTGGCGCTG
CCCAGCAAGTACGTGAATTTTCTGTACCTGGCGTCCCACTACGAAAAGCTGAAAGGCAGCCCCGAGGACAACGA
GCAGAAGCAGCTGTTCGTGGAGCAGCACAAGCATTACCTGGACGAGATAATCGAGCAAATCAGCGAGTTCAGCA
AGAGGGTGATTCTGGCCGACGCGAACCTGGATAAGGTCCTCAGCGCCTACAACAAGCACCGAGACAAACCCATC
AGGGAGCAGGCCGAGAATATCATACACCTGTTCACCCTGACAAATCTGGGCGCACCTGCGGCATTCAAATACTT
CGATACCACCATCGACAGGAAAAGGTACACTAGCACTAAGGAGGTGCTGGATGCCACCTTGATCCACCAGTCCA
TTACCGGCCTGTATGAGACCAGGATCGACCTGAGCCAGCTTGGAGGCGACTCTAGGGCGGACCCAAAAAAGAAA
AGGAAGGTGGAATTCCACCACACTGGACTAGTGGATCCGAGCTCGGTACCAAGCTTAAGTTTAAACCGCTGA

### REFERENCES

[1] Vakulskas CA, et al. A high-fidelity Cas9 mutant delivered as a ribonucleoprotein complex enables efficient gene editing in human hematopoietic stem and progenitor cells. Nat Med. 2018 August;24(8):1216-1224.
[2] Walton RT, Christie KA, Whittaker MN, Kleinstiver BP. Unconstrained genome targeting with near-PAMless engineered CRISPR-Cas9 variants. Science. 2020;368(6488):290-296.
[3] Kim HK, Yu G, Park J, et al. Predicting the efficiency of prime editing guide RNAs in human cells. Nat Biotechnol. 2021;39(2):198-206.
[4] Song M, Kim HK, Lee S, et al. Sequence-specific prediction of the efficiencies of adenine and cytosine base editors. Nat Biotechnol. 2020;38(9):1037-1043.
[5] Anzalone AV, Randolph PB, Davis JR, et al. Search-and-replace genome editing without double-strand breaks or donor DNA. Nature. 2019;576(7785):149-157.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An isolated mutant Cas9 protein, wherein the isolated mutant Cas9 protein is active in a CRISPR/Cas endonuclease system, wherein the CRISPR/Cas endonuclease system displays increased on-target editing activity relative to a wild-type CRISPR/Cas endonuclease system.

2. The isolated mutant Cas9 protein of claim 1, wherein the isolated mutant Cas9 protein comprises a substitution mutation selected from the group consisting of
(a) a single substitution mutation introduced into the WT-Cas9 protein of SEQ ID NO: 1 selected from one of the positions presented in Table 2; or
(b) a double substitution mutation introduced into the WT-Cas9 protein of SEQ ID NO: 1 selected from a mutation presented in Table 2.

3. The isolated mutant Cas9 protein of claim 1, wherein the isolated mutant Cas9 protein comprises a substitution mutation selected from the group consisting of a double substitution mutation introduced into the WT-Cas9 protein of SEQ ID NO: 1 selected from position R691A in combination with one of the mutations presented in Table 2.

4. An isolated ribonucleoprotein complex, comprising:
a mutant Cas9 protein; and
a gRNA complex,
wherein the isolated ribonucleoprotein complex is active as a CRISPR/Cas endonuclease system, wherein the resultant CRISPR/Cas endonuclease system displays increased on-target editing activity relative to a wild-type CRISPR/Cas endonuclease system.

5. The isolated ribonucleoprotein complex of claim 4, wherein the gRNA comprises a crRNA and a tracrRNA in stoichiometric (1:1) ratio.

6. The isolated ribonucleoprotein complex of claim 5, wherein the gRNA comprises:
an isolated crRNA comprises an ALT-R® crRNA directed against a specific editing target site for a given locus; and
the tracrRNA comprises an ALT-R® tracrRNA.

7. The isolated ribonucleoprotein complex of claim 5, wherein the gRNA comprises a sgRNA.

8. The isolated ribonucleoprotein complex of claim 5, wherein the mutant Cas9 protein comprises a substitution mutation selected from the group consisting of
(a) a single substitution mutation introduced into the WT-Cas9 protein of SEQ ID NO: 1 selected from one of the positions presented in Table 2; or
(b) a double substitution mutation introduced into the WT-Cas9 protein of SEQ ID NO: 1 selected from a mutation presented in Table 2.

9. The isolated ribonucleoprotein complex of claim 5, wherein the mutant Cas9 protein comprises a substitution mutation selected from the group consisting of a double substitution mutation introduced into the WT-Cas9 protein of SEQ ID NO: 1 double selected from position R691A in combination with one of the mutations presented in Table 2.

10. An isolated nucleic acid encoding a mutant Cas9 protein, wherein the mutant Cas9 protein is active in a CRISPR/Cas endonuclease system, wherein the CRISPR/Cas endonuclease system displays increased on-target editing activity relative to a wild-type CRISPR/Cas endonuclease system.

11. The isolated nucleic acid encoding a mutant Cas9 protein of claim 10, wherein the mutant Cas9 protein comprises a substitution mutation selected form the group consisting of
(a) a single substitution mutation introduced into the WT-Cas9 protein of SEQ ID NO: 1 selected from one of the positions presented in Table 2; or
(b) a double substitution mutation introduced into the WT-Cas9 protein of SEQ ID NO: 1 selected from a mutation presented in Table 2.

12. The isolated nucleic acid encoding a mutant Cas9 protein of claim 11, wherein the mutant Cas9 protein comprises a substitution mutation selected from the group consisting of a double substitution mutation introduced into the WT-Cas9 protein of SEQ ID NO: 1 selected from position R691A in combination with one of the mutations presented in Table 2.

13. A CRISPR/Cas endonuclease system comprising a mutant Cas9 protein and a gRNA, wherein the CRISPR/Cas endonuclease system displays increased on-target editing activity relative to a wild-type CRISPR/Cas endonuclease system.

14. The CRISPR/Cas endonuclease system of claim 13, wherein CRISPR/Cas endonuclease system is encoded by a DNA expression vector.

15. The CRISPR/Cas endonuclease system of claim 13, wherein DNA expression vector comprises a plasmid-borne vector.

16. The CRISPR/Cas endonuclease system of claim 13, wherein DNA expression vector is selected from a bacterial expression vector and a eukaryotic expression vector.

17. The CRISPR/Cas endonuclease system of claim 13, wherein the gRNA includes a crRNA and a tracrRNA in stoichiometric (1:1) ratio.

18. The CRISPR/Cas endonuclease system of claim 17, wherein the crRNA includes an ALT-R® crRNA directed against a specific editing target site for a given locus and the tracrRNA includes ALT-R® tracrRNA.

19. The CRISPR/Cas endonuclease system of claim 13, wherein the gRNA comprises a sgRNA.

20. The CRISPR/Cas endonuclease system of claim 13, wherein the mutant Cas9 protein comprises a substitution mutation selected from the group consisting of
(a) a single substitution mutation introduced into the WT-Cas9 protein of SEQ ID NO: 1 selected from one of the positions presented in Table 2; or
(b) a double substitution mutation introduced into the WT-Cas9 protein of SEQ ID NO: 1 selected from a mutation presented in Table 2.
